SEQUENCE LISTING (38 sequences)

SEQ ID NO: 1 - protein, 152 aa, Orthopoxvirus sp.
HKLAFNFNLEINGSDTHSTVDVYLDDSQIITFDGKDIRPTIPFMIGDEIFLPFYKNVFSEFFSLFRRVPTSTPYEDLTYFYECDYTDNKSTFDQFYLYNGEEYTVKTQEATNKNMWLTTSEFRLKKWFDGEDCIMHLRSLVRKMEDSKRNTG

SEQ ID NO: 2 - protein, 150 aa, Orthopoxvirus sp.
GHKLAFNFNLEINGSDTHSTVDVYLDDSQIITFDGKDIRPTIPFMIGDEIFLPFYKNVFSEFFSLFRRVPTSTPYEDLTYFYECDYTDNKSTFDQFYLYNGEEYTVKTQEATNKNMWLTTSEFRLKKWFDGEDCIMHLRSLVRKMEDSKR

SEQ ID NO: 3 - protein, 150 aa, Orthopoxvirus sp.
HKLVHYFNLKINGSDITNTADILLDNYPIMTFDGKDIYPSIAFMVGNKLFLDLYKNIFVEFFRLFRVSVSSQYEELEYYYSCDYTNNRPTIKQHYFYNGEEYTEIDRSKKATNKNSWLITSGFRLQKWFDSEDCIIYLRSLVRRMEDSNK

SEQ ID NO: 4 - protein, 108 aa, synthetic construct
QPVLTQPSSVSVAPGETARIPCGGDDIETKSVHWYQQKPGQAPVLVIYDDDDRPSGIPERFFGSNSGNTATLSISRVEAGDEADYYCQVWDDNNDEWVFGGGTQLTVL

SEQ ID NO: 5 - protein, 118 aa, synthetic construct
EVQLVESGGGVVQPGGSLRLSCAASGFTFSSYGMHWVRQAPGKGLEWVAFIRYDGSNKYYADSVKGRFTISRDNSKNTLYLQMNSLRAEDTAVYYCAKDRFGYYLDYWGQGTLVTVSS

SEQ ID NO: 6 - protein, 110 aa, synthetic construct
QSALTQPASVSGSPGQSITISCSGSSSNIGNNAVNWYQQLPGKAPKLLIYYDDLLPSGVSDRFSGSKSGTSAFLAISGLQSEDEADYYCAAWDDSLNGPVFGGGTKLTVL

SEQ ID NO: 7 - protein, 121 aa, synthetic construct
QVQLVESGGGLVKPGGSLRLSCAASGFTFSSYGMHWVRQAPGKGLEWVAFIRYDGSNKYYADSVKGRFTISRDNSKNTLYLQMNSLRAEDTAVYYCAKDRGLGDGTYFDYWGQGTTVTVSS

SEQ ID NO: 8 - protein, 241 aa, synthetic construct
QPVLTQPSSVSVAPGETARIPCGGDDIETKSVHWYQQKPGQAPVLVIYDDDDRPSGIPERFFGSNSGNTATLSISRVEAGDEADYYCQVWDDNNDEWVFGGGTQLTVLGGGGSGGGGSGGGGSEVQLVESGGGVVQPGGSLRLSCAASGFTFSSYGMHWVRQAPGKGLEWVAFIRYDGSNKYYADSVKGRFTISRDNSKNTLYLQMNSLRAEDTAVYYCAKDRFGYYLDYWGQGTLVTVSS

SEQ ID NO: 9 - protein, 241 aa, synthetic construct
EVQLVESGGGVVQPGGSLRLSCAASGFTFSSYGMHWVRQAPGKGLEWVAFIRYDGSNKYYADSVKGRFTISRDNSKNTLYLQMNSLRAEDTAVYYCAKDRFGYYLDYWGQGTLVTVSSGGGGSGGGGSGGGGSQPVLTQPSSVSVAPGETARIPCGGDDIETKSVHWYQQKPGQAPVLVIYDDDDRPSGIPERFFGSNSGNTATLSISRVEAGDEADYYCQVWDDNNDEWVFGGGTQLTVL

SEQ ID NO: 10 - protein, 246 aa, synthetic construct
QSALTQPASVSGSPGQSITISCSGSSSNIGNNAVNWYQQLPGKAPKLLIYYDDLLPSGVSDRFSGSKSGTSAFLAISGLQSEDEADYYCAAWDDSLNGPVFGGGTKLTVLGGGGSGGGGSGGGGSQVQLVESGGGLVKPGGSLRLSCAASGFTFSSYGMHWVRQAPGKGLEWVAFIRYDGSNKYYADSVKGRFTISRDNSKNTLYLQMNSLRAEDTAVYYCAKDRGLGDGTYFDYWGQGTTVTVSS

SEQ ID NO: 11 - protein, 246 aa, synthetic construct
QVQLVESGGGLVKPGGSLRLSCAASGFTFSSYGMHWVRQAPGKGLEWVAFIRYDGSNKYYADSVKGRFTISRDNSKNTLYLQMNSLRAEDTAVYYCAKDRGLGDGTYFDYWGQGTTVTVSSGGGGSGGGGSGGGGSQSALTQPASVSGSPGQSITISCSGSSSNIGNNAVNWYQQLPGKAPKLLIYYDDLLPSGVSDRFSGSKSGTSAFLAISGLQSEDEADYYCAAWDDSLNGPVFGGGTKLTVL

SEQ ID NO: 12 - protein, 111 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVA

SEQ ID NO: 13 - protein, 119 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA

SEQ ID NO: 14 - protein, 245 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAGGGGSGGGGSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA

SEQ ID NO: 15 - protein, 412 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAGGGGSGGGGSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAGGGGSGGGGSGGGGSHKLAFNFNLEINGSDTHSTVDVYLDDSQIITFDGKDIRPTIPFMIGDEIFLPFYKNVFSEFFSLFRRVPTSTPYEDLTYFYECDYTDNKSTFDQFYLYNGEEYTVKTQEATNKNMWLTTSEFRLKKWFDGEDCIMHLRSLVRKMEDSKRNTG

SEQ ID NO: 16 - protein, 501 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAGGGGSGGGGSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKHHHHHHHH

SEQ ID NO: 17 - protein, 243 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 18 - protein, 420 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAGGGGSGGGGSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAGGGGSGGGGSGGGGSHKLAFNFNLEINGSDTHSTVDVYLDDSQIITFDGKDIRPTIPFMIGDEIFLPFYKNVFSEFFSLFRRVPTSTPYEDLTYFYECDYTDNKSTFDQFYLYNGEEYTVKTQEATNKNMWLTTSEFRLKKWFDGEDCIMHLRSLVRKMEDSKRNTGHHHHHHHH

SEQ ID NO: 19 - protein, 504 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAGGGGSGGGGSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAGGGGSQVQLVESGGGLVKPGGSLRLSCAASGFTFSSYGMHWVRQAPGKGLEWVAFIRYDGSNKYYADSVKGRFTISRDNSKNTLYLQMNSLRAEDTAVYYCAKDRGLGDGTYFDYWGQGTTVTVSSGGGGSGGGGSGGGGSQSALTQPASVSGSPGQSITISCSGSSSNIGNNAVNWYQQLPGKAPKLLIYYDDLLPSGVSDRFSGSKSGTSAFLAISGLQSEDEADYYCAAWDDSLNGPVFGGGTKLTVLHHHHHHHH

SEQ ID NO: 20 - protein, 499 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAGGGGSGGGGSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAGGGGSEVQLVESGGGVVQPGGSLRLSCAASGFTFSSYGMHWVRQAPGKGLEWVAFIRYDGSNKYYADSVKGRFTISRDNSKNTLYLQMNSLRAEDTAVYYCAKDRFGYYLDYWGQGTLVTVSSGGGGSGGGGSGGGGSQPVLTQPSSVSVAPGETARIPCGGDDIETKSVHWYQQKPGQAPVLVIYDDDDRPSGIPERFFGSNSGNTATLSISRVEAGDEADYYCQVWDDNNDEWVFGGGTQLTVLHHHHHHHH

SEQ ID NO: 21 - protein, 232 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 232 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 232 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 24 - protein, 232 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCLVKGFYPSDIAVEWESNGQPENNYKTTPPVLDSDGSFALYSKLTVDKSRWQQGNVFSCSVMHEALHNHYTQKSLSLSPGK

SEQ ID NO: 25 - protein, 232 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTWPPVLDSDGSFFLTSKLTVDKSRWQQGNVFSCSVMHEALHNHYTQKSLSLSPGK

SEQ ID NO: 26 - protein, 232 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 232 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSFLTVDKSRWQQGNVFSCSVMHEALHNHYTQKSLSLSPGK

SEQ ID NO: 28 - protein, 234 aa, synthetic construct
EPKSSDKTHTCPPCPAPELLGGPSVFLFPPKPKDTLMISRTPEVTCWVDVSHEDPEVKFNWYVDGVEVHNAKTKPREEQYNSTYRVVSVLTVLHQDWLNGKEYKCKVSNKALPAPIEKTISKAKGQPFRPEVHLLPPSREEMTKNQVSLTCLARGFYPKDIAVEWESNGQPENNYKTTPSRQEPSQGTTTFAVTSKLTVDKSRWQQGNVFSCSVMHEALHNHYTQKTISLSPGK

SEQ ID NO: 29 - protein, 235 aa, synthetic construct
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPPSEELALNELVTLTCLVKGFYPSDIAVEWLQGSQELPREKYLTWAPVLDSDGSFFLYSILRVAAEDWKKGDTFSCSVMHEALHNHYTQKSLDRSPGK

SEQ ID NO: 30 - protein, 232 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTSPPSRDELTKNQVSLRCHVKGFYPSDIAVEWESNGQPENNYKTTKPVLDSDGSFFLYSKLTVDKSRWQQGNVFSCSVMHEALHNHYTQKSLSLSPGK

SEQ ID NO: 31 - protein, 399 aa, synthetic construct
HKLAFNFNLEINGSDTHSTVDVYLDDSQIITFDGKDIRPTIPFMIGDEIFLPFYKNVFSEFFSLFRRVPTSTPYEDLTYFYECDYTDNKSTFDQFYLYNGEEYTVKTQEATNKNMWLTTSEFRLKKWFDGEDCIMHLRSLVRKMEDSKRNTGGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCLVKGFYPSDIAVEWESNGQPENNYKTTPPVLDSDGSFALYSKLTVDKSRWQQGNVFSCSVMHEALHNHYTQKSLSLSPGK

SEQ ID NO: 32 - protein, 492 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAGGGGSGGGGSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTWPPVLDSDGSFFLTSKLTVDKSRWQQGNVFSCSVMHEALHNHYTQKSLSLSPGK

SEQ ID NO: 33 - protein, 659 aa, synthetic construct
HKLAFNFNLEINGSDTHSTVDVYLDDSQIITFDGKDIRPTIPFMIGDEIFLPFYKNVFSEFFSLFRRVPTSTPYEDLTYFYECDYTDNKSTFDQFYLYNGEEYTVKTQEATNKNMWLTTSEFRLKKWFDGEDCIMHLRSLVRKMEDSKRNTGGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAGGGGSGGGGSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA

SEQ ID NO: 34 - protein, 659 aa, synthetic construct
HKLAFNFNLEINGSDTHSTVDVYLDDSQIITFDGKDIRPTIPFMIGDEIFLPFYKNVFSEFFSLFRRVPTSTPYEDLTYFYECDYTDNKSTFDQFYLYNGEEYTVKTQEATNKNMWLTTSEFRLKKWFDGEDCIMHLRSLVRKMEDSKRNTGGGGGSGGGGSGGGGSDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAGGGGSGGGGSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCLVKGFYPSDIAVEWESNGQPENNYKTTPPVLDSDGSFALYSKLTVDKSRWQQGNVFSCSVMHEALHNHYTQKSLSLSPGK

SEQ ID NO: 35 - protein, 232 aa, synthetic construct; identical to SEQ ID NO: 25

SEQ ID NO: 36 - protein, 659 aa, synthetic construct
HKLAFNFNLEINGSDTHSTVDVYLDDSQIITFDGKDIRPTIPFMIGDEIFLPFYKNVFSEFFSLFRRVPTSTPYEDLTYFYECDYTDNKSTFDQFYLYNGEEYTVKTQEATNKNMWLTTSEFRLKKWFDGEDCIMHLRSLVRKMEDSKRNTGGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTSPPSRDELTKNQVSLRCHVKGFYPSDIAVEWESNGQPENNYKTTKPVLDSDGSFFLYSKLTVDKSRWQQGNVFSCSVMHEALHNHYTQKSLSLSPGKGGGGSGGGGSGGGGSDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAGGGGSGGGGSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA

SEQ ID NO: 37 - protein, 5 aa, synthetic construct
GGGGS

SEQ ID NO: 38 - protein, 8 aa, synthetic construct
HHHHHHHH

(19) United States
(12) Patent Application Publication
(10) Pub. No.: US 2026/0008822 A1
(43) Pub. Date: Jan. 8, 2026

(54) BISPECIFIC FUSION PROTEIN USING ORTHOPOXVIRUS MAJOR HISTOCOMPATIBILITY COMPLEX (MHC) CLASS I-LIKE PROTEIN (OMCP) AND TUMOR-SPECIFIC BINDING PARTNER

(71) Applicant: Recourse Biologics, Inc., Houston, TX (US)

(72) Inventors: Alexander Sasha KRUPNICK, Owings Mills, MD (US); Eric Reed LAZEAR, Carrboro, NC (US); Sarah HEIN, Houston, TX (US); Daniel Marvin WATKINS, Houston, TX (US)

(73) Assignee: Recourse Biologics, Inc., Houston, TX (US)

(21) Appl. No.: 19/044,533

(22) Filed: Feb. 3, 2025

Related U.S. Application Data
(63) Continuation of application No. 17/431,664, filed on Aug. 17, 2021, now abandoned.
(60) Provisional application No. 62/807,190, filed on Feb. 18, 2019.

(51) Int. Cl. C07K 14/005; A61K 39/00; A61P 35/00; C07K 16/30

(52) U.S. Cl. CPC C07K 14/005; A61P 35/00; C07K 16/3015; C07K 16/3023; C07K 16/303; C07K 16/3038; C07K 16/3046; C07K 16/3053; C07K 16/3061; C07K 16/3069; A61K 39/00; C07K 2317/31; C07K 2319/30

(57) ABSTRACT

Therapeutic polypeptides, compositions thereof and methods of use thereof for activating NK cells and treating tumors are provided. The therapeutic polypeptides can include a first domain for binding NKG2D and a second domain for binding a tumor target.

### CROSS-REFERENCE TO RELATED APPLICATIONS

The present application claims priority to U.S. Provisional Application 62/807,190, filed Feb. 18, 2019, the entirety of which is incorporated herein by reference.

### REFERENCE TO A SEQUENCE LISTING XML

The instant application contains a Sequence Listing which has been submitted electronically in XML format and is hereby incorporated by reference in its entirety. Said XML file, created on Sep. 16, 2025, is named 794516_000060_SL.xml and is 44,121 bytes in size.

### INCORPORATION BY REFERENCE

WO2016/100375 and WO2017/136818 are incorporated by reference herein in their entirety. Moreover, all publications referenced herein are incorporated by reference herein in their entirety.

### BACKGROUND

Bispecific lymphocyte engagers work by engaging both the cytotoxic lymphocyte and the tumor cell simultaneously. This creates an artificial immune synapse and increases the efficiency of immune engagement and destruction of the cancer cells. The ligands in these bispecific proteins are derived from engineered targeted antibodies expressed together in such a way as to form a continuous therapeutic protein. These therapeutic proteins can be effective in cancer therapeutics.

Current bispecific therapeutics in development function via engagement of CD3, which is part of the T cell receptor complex. CD3 is expressed on cytotoxic CD8⁺ T cells, and helper CD4⁺ T cells. Broad CD3 engagement may result in non-specific T cell activation away from the tumor site, leading to toxicities, including “cytokine storm.” More recently, some groups have begun targeting NK cells via the NKp46 or CD16 receptor.

NKG2D expression is unique, however, because it is constitutively expressed on human CD8⁺ T cells and NK cells. Therefore, NKG2D targeting would enable engagement of both the innate and adaptive cytotoxic lymphocytes (CD8⁺ T cells and NK cells), resulting in a more robust anti-tumor activity.

### SUMMARY

The present disclosure relates to a new class of bispecific (or multi-specific) therapeutic proteins which targets both the NK and CD8⁺ T cells via a ligand to the NKG2D receptor on one side, and a binding partner directed to a tumor-specific target on the other side. The NKG2D receptor can be bound via any specific ligand, such as a mono or polyclonal antibody, the Orthopoxvirus Major Histocompatibility complex (MHC) class I-like protein (OMCP) ligand, or a native NKG2D ligand. The tumor target can be selected from any cell-surface target which is either specifically expressed in cancer cells or which has increased expression in cancer cells compared to normal tissues.

In some embodiments, a polypeptide is provided that includes a first domain and a second domain, where the first domain includes a first amino acid sequence of at least 80% homology to SEQ ID NOs: 1, 2 or 3 and is capable of binding human NKG2D with a binding affinity of about 0.01 nM to about 1000 nM, where the second domain includes a second amino acid sequence capable of binding to a peptide on a tumor cell, where the peptide is either specific to the tumor cell or overexpressed on the tumor cell compared to a non-tumor cell of the same tissue origin as the tumor cell.

In some embodiments, a polypeptide of the present disclosure can include a first domain and a second domain, wherein the first domain comprises a first amino acid sequence of at least 80% homology to amino acid positions 48 to 67 and 110 to 147 of SEQ ID NO: 1, and where the second domain can include a second amino acid sequence capable of binding to a peptide on a tumor cell, where the peptide either specific to the tumor cell or overexpressed on the tumor cell compared to a non-tumor cell of the same tissue origin as the tumor cell.

In some embodiments, a polypeptide of the present disclosure can include a first domain and a second domain, wherein the first domain comprises a first amino acid sequence of at least 80% homology to amino acid positions 49 to 68 and 111 to 148 of SEQ ID NO: 2, and where the second domain can include a second amino acid sequence capable of binding to a peptide on a tumor cell, where the peptide either specific to the tumor cell or overexpressed on the tumor cell compared to a non-tumor cell of the same tissue origin as the tumor cell.

In some embodiments, a polypeptide of the present disclosure can include a first domain and a second domain, wherein the first domain comprises a first amino acid sequence of at least 80% homology to amino acid positions 48 to 66 and 111 to 148 of SEQ ID NO: 3, and where the second domain can include a second amino acid sequence capable of binding to a peptide on a tumor cell, where the peptide either specific to the tumor cell or overexpressed on the tumor cell compared to a non-tumor cell of the same tissue origin as the tumor cell.

In some embodiments, a pharmaceutical composition is provided that includes a polypeptide of the present disclosure.

In some embodiments, a method is provided for treating a tumor in a patient by administering a pharmaceutical composition of the present disclosure that includes a polypeptide of the present disclosure to the patient.

### BRIEF DESCRIPTION OF THE DRAWINGS

FIG. 1 depicts an exemplary bispecific polypeptide of the present disclosure that includes OMCP (circle) linked to anti anti-tumor target, specifically a single-chain variable fragment (scFv) that includes a variable heavy chain and variable light chain directed to a tumor target.

FIG. 2A depicts an exemplary tri-specific polypeptide of the present disclosure that includes two single chain variable fragments (scFv), α-tumor target 1 and α-tumor target 2 that can bind to a first tumor target and a second tumor target, linked to an antibody Fc domain that is also linked to two single chain variable fragments that can bind NKG2D (α-NKG2D).

FIG. 2B depicts an exemplary tri-specific polypeptide of the present disclosure that includes two single chain variable fragments (scFv), α-tumor target 1 and α-tumor target 2 that can bind to a first tumor target and a second tumor target, linked to an antibody Fc domain that is also linked to two NKG2D ligands (“OMCP”).

FIG. 2C depicts an exemplary tri-specific polypeptide of the present disclosure that includes two single chain variable fragments (scFv), α-tumor target 1 and α-tumor target 2 that can bind to a first tumor target and a second tumor target, linked to an antibody Fc domain that is also linked to six NKG2D ligands (“OMCP”).

FIG. 3A depicts an exemplary quad-specific polypeptide of the present disclosure that includes two single chain variable fragments (scFv), α-tumor target 1 and α-tumor target 2 that can bind to a first tumor target and a second tumor target, linked to an antibody Fc domain that is also linked to a NKG2D ligand (“OMCP”) and a single chain variable fragment that can bind CD3e (“α-CD3e T cell target”).

FIG. 3B depicts an exemplary quad-specific polypeptide of the present disclosure that includes two single chain variable fragments (scFv), α-tumor target 1 and α-tumor target 2 that can bind to a first tumor target and a second tumor target, linked to an antibody Fc domain that is also linked to a NKG2D ligand (“OMCP”) and a single chain variable fragment that can bind FCGH1 (“α-FCGH1 NK cell target”).

FIG. 4A depicts exemplary bi-specific polypeptides of the present disclosure with a cytokine (38A 42K IL2) in addition to a scFV directed to a tumor target (α-tumor target) and a NKG2D ligand (“OMCP”) where there is either a linker between the NKG2D ligand and the cytokine (left structure) or two linkers, one between the cytokine and the scFv and one between the cytokine and the NKG2D ligand (right structure).

FIG. 4B depicts an exemplary polypeptide of the present disclosure which includes two scFv's, α-tumor target 1 and α-tumor target 2, connected to an antibody Fc domain that is further linked to a NKG2D ligand (“OMCP”) and a cytokine (38A 42K IL2).

FIG. 5A depicts an exemplary bi-specific scFv of the present disclosure.

FIG. 5B depicts an exemplary bi-specific scFv of the present disclosure.

FIG. 5C depicts an exemplary bi-specific scFv of the present disclosure.

FIG. 5D depicts an exemplary bi-specific scFv of the present disclosure.

FIG. 5E depicts linear schematics of the exemplary bi-specific scFv's of FIGS. 5A-5D.

FIG. 6A depicts an exemplary tri-specific scFv of the present disclosure which includes a Fc portion.

FIG. 6B depicts an exemplary tri-specific scFv of the present disclosure which includes a Fc portion.

FIG. 6C depicts an exemplary tri-specific scFv of the present disclosure which includes a Fc portion.

FIG. 6D depicts an exemplary tri-specific scFv of the present disclosure which includes a Fc portion.

FIG. 7A depicts plasmon resonance measurements for E0 binding to NKG2D.

FIG. 7B depicts plasmon resonance measurements for E1 binding to NKG2D.

FIG. 7C depicts plasmon resonance measurements for E2 binding to NKG2D.

FIG. 7D depicts plasmon resonance measurements for E3 binding to NKG2D.

FIG. 8A depicts plasmon resonance measurements for E0 binding to EGFR-Fc.

FIG. 8B depicts plasmon resonance measurements for E1 binding to EGFR-Fc.

FIG. 8C depicts plasmon resonance measurements for E2 binding to EGFR-Fc.

FIG. 8D depicts plasmon resonance measurements for E3 binding to EGFR-Fc.

FIG. 9 depicts the cell viability for each treatment as a function of concentration of bi-specific polypeptides of the present disclosure in Example 2.

FIG. 10 depicts the cell viability for each treatment at 1×10⁻⁸ M of the bi-specific polypeptides in Example 2.

FIG. 11 depicts the cell viability for each treatment at 1×10⁻⁹ M of the bi-specific polypeptides in Example 2.

FIG. 12 shows images of the cells for the negative control (no construct added) and the treatment groups in the 1 nM group for Example 2.

FIG. 13 depicts the cell viability for each treatment at 1×10⁻¹⁰ M of the bi-specific polypeptides in Example 2.

FIG. 14 depicts the % dead cells treated with bi-specific polypeptides in Example 3 at 1×10⁻⁸ M.

FIG. 15 depicts the % dead cells treated with bi-specific polypeptides in Example 3 at 1×10⁻¹⁰ M.

FIG. 16 depicts the % dead cells treated with bi-specific polypeptides in Example 3 at 1×10⁻¹² M.

FIG. 17 depicts the % dead cells treated with bi-specific polypeptides in Example 4 at 100 pM in the presence of NK cells. The controls are tumor cells with or without the NK cells.

FIG. 18 depicts the % dead cells treated with bi-specific polypeptides in Example 4 at 100 pM in the presence of T cells. The controls are tumor cells with or without the CD8⁺ T cells.

FIG. 19A depicts cytokine production (IFN-γ) with or without polypeptides E0, E1, E2 and E3 at different concentrations of E0, E1, E2 and E3 with PBMCs only or with PBMCs and tumor cells.

FIG. 19B depicts cytokine production (TNF-α) with or without polypeptides E0, E1, E2 and E3 at different concentrations of E0, E1, E2 and E3 with PBMCs only or with PBMCs and tumor cells.

FIG. 19C depicts cytokine production (IL-6) with or without polypeptides E0, E1, E2 and E3 at different concentrations of E0, E1, E2 and E3 with PBMCs only or with PBMCs and tumor cells.

FIG. 19D depicts cytokine production (IL-17a) with or without polypeptides E0, E1, E2 and E3 at different concentrations of E0, E1, E2 and E3 with PBMCs only or with PBMCs and tumor cells.

### DETAILED DESCRIPTION

The present disclosure provides a therapeutic fusion peptide comprising, in one embodiment, a NKG2D ligand fused to an tumor-targeted peptide, such as an anti-tumor antibody, with or without a connecting linker in the middle. The NKG2D ligand could be selected from either full-length OMCP, truncated OMCP, an anti-NKG2D antibody, or an MHC class I-related glycoprotein as disclosed in more detail herein. The tumor-targeted peptide is either a full-length or variable-region domain of an antibody targeted against any protein which has tumor-specific mutations or which is more highly expressed at the surface of tumor cells than normal tissues. In another embodiment, the tumor-targeted protein is a form of a naturally occurring ligand to the targeted protein. In an embodiment using a natural ligand to a target, the ligand may be mutated to either increase or decrease affinity of the ligand to the targeted protein. Non-limiting examples of tumor-selective targets are ERBB2, CD19, EPCAM, MS4A1, FOLH1, CEACAM5, IL3RA, PMEL, CLEC12A, KDR, EGFR, TAG-72 (tumor associated glycoprotein 72) disialoganglioside GD2, CD20, CD123, CD33, BCMA, CD38, B7H3/CD276, GPA33,SSTR2, GPC3, and CDH3.

It should be understood that the term “peptide,” “polypeptide,” or “protein” can be used interchangeably herein to refer to a single peptide with multiple sequences that each give rise to specific functions (such as an NKG2D receptor ligand or a binding partner of a tumor-specific target) or may refer to one or more peptides bound or otherwise complexed together (such as a heterodimer where the NKG2D receptor ligand and binding partner of a tumor-specific target are on separate peptides that complex together, for example, where the NKG2D receptor ligand is linked to a first Fc portion and the binding partner of a tumor-specific target is linked to the second Fc portion, where the first and second Fc portions complex together to form a Fc antibody domain).

As used herein, the singular forms “a”, “an” and “the” include plural referents unless the context clearly dictates otherwise.

The use of the term “or” in the claims and the present disclosure is used to mean “and/or” unless explicitly indicated to refer to alternatives only or the alternatives are mutually exclusive.

Use of the term “about”, when used with a numerical value, is intended to include +/−10%. By way of example but not limitation, if a number of amino acids is identified as about 200, this would include 180 to 220 (plus or minus 10%).

The term “subject” or “patient” refers to a mammalian subject to be treated, with human patients being preferred. In some cases, the methods of the invention find use in experimental animals, in veterinary application, and in the development of animal models for disease, including, but not limited to, rodents including mice, rats, and hamsters, and primates.

The therapeutic fusion peptides of the present disclosure can be used as a therapeutic to treat malignant tumors and cancers. Non-limiting examples of cancers to be treated in embodiments of the present disclosure include solid tumors such as breast cancer, prostate cancer, melanoma, ovarian cancer, gastric cancer, glioblastoma, neuroblastoma and lung cancer; it also includes hematological cancers such B cell lymphoma, diffuse large cell B cell lymphoma, lymphoblastic leukemia, lymphocytic leukemia, and follicular lymphoma. In any embodiment of the disclosure, the tumor cell can be selected from the group consisting of a breast cancer cell, a prostate cancer cell, a melanoma cell, an ovarian cancer cell, a gastric cancer cell, a glioblastoma cell, a neuroblastoma cell, a lung cancer cell, a lymphoma cell, a leukemia cell, a colon cancer cell, a renal cell carcinoma, a pancreatic cancer cell, and a hepatocellular carcinoma cell.

### Bispecific Fusion Proteins

In some embodiments, a polypeptide of the present disclosure can include a first domain and a second domain, where the first domain can include a first amino acid sequence that is capable of binding to human NKG2D, where the second domain can include a second amino acid sequence that is capable of binding to a peptide on a tumor cell, where the peptide is either specific to the tumor cell or overexpressed on the tumor cell compared to a non-tumor cell of the same tissue origin as the tumor cell. By way of example, but not limitation, the first amino acid sequence that is capable of binding to human NKG2D can be a human NKG2D ligand or an antibody that can bind human NKG2D. By way of further example, but not limitation, the NKG2D ligand can be OMCP or a variant or derivative thereof that can bind to human NKG2D. In some embodiments, the human NKG2D ligand can bind human NKG2D with a binding affinity of about 0.01 nM to about 1000 nM.

In some embodiments, a polypeptide of the present disclosure can include a first domain and a second domain, where the first domain can include a first amino acid sequence that has at least 80% homology to any of SEQ ID NOs: 1-3 and is capable of binding to human NKG2D with a binding affinity of about 0.01 nM to about 1000 nM, where the second domain can include a second amino acid sequence that is capable of binding to a peptide on a tumor cell, where the peptide is either specific to the tumor cell or overexpressed on the tumor cell compared to a non-tumor cell of the same tissue origin as the tumor cell.

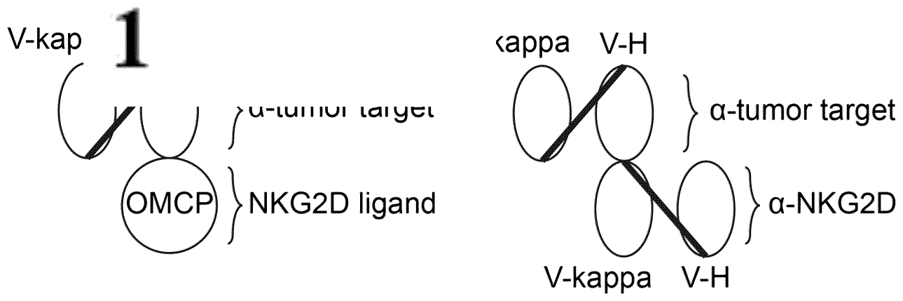

By way of example, but not limitation, as shown in FIG. 1, a bispecific polypeptide of the present disclosure can include OMCP-a NKG2D ligand-and an anti-tumor single-chain variable fragment (scFv) derived from an antibody to the peptide on a tumor cell.

In some embodiments, a polypeptide of the present disclosure can include a first domain and a second domain, wherein the first domain comprises a first amino acid sequence of at least 80% homology to amino acid positions 48 to 67 and 110 to 147 of SEQ ID NO: 1, and where the second domain can include a second amino acid sequence capable of binding to a peptide on a tumor cell, where the peptide either specific to the tumor cell or overexpressed on the tumor cell compared to a non-tumor cell of the same tissue origin as the tumor cell.

In some embodiments, a polypeptide of the present disclosure can include a first domain and a second domain, wherein the first domain comprises a first amino acid sequence of at least 80% homology to amino acid positions 49 to 68 and 111 to 148 of SEQ ID NO: 2, and where the second domain can include a second amino acid sequence capable of binding to a peptide on a tumor cell, where the peptide either specific to the tumor cell or overexpressed on the tumor cell compared to a non-tumor cell of the same tissue origin as the tumor cell.

In some embodiments, a polypeptide of the present disclosure can include a first domain and a second domain, wherein the first domain comprises a first amino acid sequence of at least 80% homology to amino acid positions 48 to 66 and 111 to 148 of SEQ ID NO: 3, and where the second domain can include a second amino acid sequence capable of binding to a peptide on a tumor cell, where the peptide either specific to the tumor cell or overexpressed on the tumor cell compared to a non-tumor cell of the same tissue origin as the tumor cell.

### NKG2D Ligands

Ligands that bind to NKG2D share an MHC class I-related ala2 superdomain that constitutes the common site for interaction with NKG2D. Non-limiting examples of ligands that bind to NKG2D include MHC class I-related glycoproteins such as MIC family proteins (i.e., MICA, MICB), UL16-binding family proteins (i.e., ULBP1, ULBP2, ULPB3, ULBP4, ULBP5, ULBP6), retinoid acid early induce gene 1 (Rae1)-like proteins (i.e., Rae1α, Rae1β Rae1γ,Rae1δ, Rae1ε), members of the H60 protein family (i.e., H60a, H60b, H60c), h-HLA-A, as well as Mult1 in mice and orthopoxvirus major histocompatibility complex class I-like protein (OMCP).

In some embodiments, the NKG2D ligand can be a MHC class-I-related glycoprotein. In some embodiments, the NKG2D ligand can be selected from the group consisting of MICA, MICB, ULBP1, ULBP2, ULBP3, ULBP4, ULBP5, ULBP6, Rae1α, Rae1β Rae1γ, Rae1δ,Rae1ε, H60a, H60b, H60c, h-HLA-A, Mult1 and OMCP. In some embodiments, the NKG2D ligand can be a UL16-binding family protein or a MIC family protein. In some embodiments, the NKG2D ligand can be selected from the group consisting of ULBP1, ULBP2, ULBP3, ULBP4, ULBP5, and ULBP6. In some embodiments, a NKG2D ligand can be ULBP3. In some embodiments, the first domain of the polypeptide includes a NKG2D ligand that is OMCP or a variant thereof.

A variant of OMCP can be a truncated or mutated OMCP that has about the same binding affinity of the full length OMCP. In an embodiment, a variant of OMCP can be a truncated or mutated OMCP that has a slightly lower binding affinity relative to the binding affinity of the full length OMCP. In another embodiment, a variant of OMCP can be a truncated or mutated OMCP that has a higher binding affinity relative to the binding affinity of the full length OMCP.

Methods to determine binding affinity of a ligand to target protein are known in the art and described above. For example, as described below in the examples, binding affinity can be determined by measuring surface plasmon resonance. OMCP specifically binds to NKG2D with a binding affinity of about 0.1 to about 5 nM. For example, OMCP specially binds to human NKG2D with a binding affinity of about 0.2 nM and mouse NKG2D with a binding affinity of about 3 nM.

In a preferred embodiment, the NKG2D ligand is OMCP or a variant thereof that binds to human NKG2D with a binding affinity of about 1000 nM to about 0.01 nM. In certain embodiments, OMCP or a variant thereof binds to human NKG2D with a binding affinity of about 100 nM to about 0.01 nM, about 10 nM to about 0.01 nM, or about 1 nM to about 0.01 nM. In other embodiments, OMCP or a variant thereof binds to human NKG2D with a binding affinity of about 1000 nM to about 1 nM, or about 1000 nM to about 10 nM, or about 1000 nM to about 100 nM. In still other embodiments, OMCP or a variant thereof binds to human NKG2D with a binding affinity of about 100 nM to about 1 nM, or about 100 nM to 10 nM. For example, the OMCP or a variant thereof can bind to human NKG2D with a binding affinity of about 1000 nM, about 500 nM, about 100 nM, about 50 nM, about 10 nM, about 9 nM, about 8 nM, about 7 nM, about 6 nM about 5 nM, about 4 nM, about 3 nM, about 2 nM, about 1 nM, about 0.9 nM, about 0.8 nM, about 0.7 nM, about 0.6 nM, about 0.5 nM, about 0.4 nM, about 0.3 nM, about 0.2 nM or about 0.1 nM. By way of further example, but not limitation, the OMCP or a variant thereof can bind to human NKG2D with a binding affinity of about 1000 nM to about 0.1 nM, about 100 nM to about 0.1 nM, about 10 nM to about 0.1 nM, or about 1 nM to about 0.1 nM. It should be understood that the foregoing disclosure regarding binding affinities can apply to a first amino acid sequence of polypeptides of the disclosure and any other NKG2D binding domain of the polypeptides of the present disclosure, such as, by way of example not limitation, the first amino acid sequences with homology to regions of SEQ ID NOs: 1-3.

The sequence information for the full length OMCP amino acid sequence can be found using, for example, the GenBank accession number 4FFE_Z, 4FFE_Y or 4FFE_X. A skilled artisan will appreciate that homologs of OMCP may be found in other species or viruses. For example, see Lefkowitz et al, Nucleic Acids Res 2005; 33: D311-316, which is herein incorporated by reference in its entirety, which describes eighteen OMCP variants between cowpox and monkeypox virus strains. In an embodiment, OMCP is from an orthopoxvirus. In a specific embodiment, OMCP is from a cowpox virus or a monkeypox virus. In another specific embodiment, OMCP is from the Brighton Red strain of cowpoxvirus. Homologs can be found in other species by methods known in the art. For example, sequence similarity may be determined by conventional algorithms, which typically allow introduction of a small number of gaps in order to achieve the best fit. In particular, “percent identity” of two polypeptides or two nucleic acid sequences is determined using the algorithm of Karlin and Altschul (Proc. Natl. Acad. Sci. USA 87:2264-2268, 1993). Such an algorithm is incorporated into the BLASTN and BLASTX programs of Altschul et al. (J. Mol. Biol. 215:403-410, 1990). BLAST nucleotide searches may be performed with the BLASTN program to obtain nucleotide sequences homologous to a nucleic acid molecule of the invention. Equally, BLAST protein searches may be performed with the BLASTX program to obtain amino acid sequences that are homologous to a polypeptide of the invention. To obtain gapped alignments for comparison purposes, Gapped BLAST is utilized as described in Altschul et al. (Nucleic Acids Res. 25:3389-3402, 1997). When utilizing BLAST and Gapped BLAST programs, the default parameters of the respective programs (e.g., BLASTX and BLASTN) are employed. See www.ncbi.nlm.nih.gov for more details. Generally a homolog will have a least 80, 81, 82, 83, 84, 85, 86, 87, 88, or 89% homology. In any of the foregoing embodiments of the disclosure, the sequence can be at least 80, 81, 82, 83, 84, 85, 86, 87, 88, 89, 90, 91, 92, 93, 94, 95, 96, 97, 98, 99, or 100% homologous to OMCP, such as that of any of SEQ ID NOs: 1-3. Generally, OMCP from known strains of monkeypox and cowpox are all within about 97% homology.

A skilled artisan will appreciate that structural homologs of OMCP may be found in other species or viruses. A structural homolog may be a protein that is structurally related but the sequence is a distal homolog. For example, OMCP has low sequence identity for endogenous NKG2D ligands however it was discovered that OMCP would bind to NKG2D based on structural homology. Structural homologs can be found in other species by methods known in the art. For example, protein structure prediction may be determined by various databases, such as Phyre and Phyre2. Such databases generate reliable protein models that may be used to determine structural homologs. The main results table in Phyre2 provides confidence estimates, images and links to the three-dimensional predicted models and information derived from either Structural Classification of Proteins database (SCOP) or the Protein Data Bank (PDB) depending on the source of the detected template. For each match a link takes the user to a detailed view of the alignment between the user sequence and the sequence of known three-dimensional structure. See www.sbg.bio.ic.ac.uk/phyre2/for more details. Generally, a structural homolog will have a least 50, 51, 52, 53, 54, 55, 56, 57, 58, or 59% confidence with OMCP. In an embodiment, a structural homolog will have a least 60, 61, 62, 63, 64, 65, 66, 67, 68, or 69% confidence with OMCP. In another embodiment, a structural homolog will have a least 70, 71, 72, 73, 74, 75, 76, 77, 78, or 79% confidence with OMCP. In still another embodiment, a structural homolog will have a least 80, 81, 82, 83, 64, 85, 86, 87, 88, or 89% confidence with OMCP. In still yet another embodiment, a structural homolog may have at least 90, 91, 92, 93, 94, 95, 96, 97, 98, 99, or 100% confidence with OMCP. The structural information for OMCP-human NKG2D may be found using the PDB ID: 4PDC. It should be understood that such structural homologs can be the first amino acid sequence of polypeptides of the present disclosure and any other NKG2D binding domain of the polypeptides of the present disclosure.

In any of the foregoing embodiments, first amino acid sequence can be a sequence of OMCP such as the sequences set forth in SEQ ID NO: 1 (HKLAFNFNLEINGSDTHSTVDVYLDDSQIITFDGKDIRPTIPFMIGDEIFLPFYKNVFSEFF SLFRRVPTSTPYEDLTYFYECDYTDNKSTFDQFYLYNGEEYTVKTQEATNKNMWLTTSE FRLKKWFDGEDCIMHLRSLVRKMEDSKRNTG), SEQ ID NO: 2 (GHKLAFNFNLEINGSDTHSTVDVYLDDSQIITFDGKDIRPTIPFMIGDEIFLPFYKNVFSEF FSLFRRVPTSTPYEDLTYFYECDYTDNKSTFDQFYLYNGEEYTVKTQEATNKNMWLTTS EFRLKKWFDGEDCIMHLRSLVRKMEDSKR), and SEQ ID NO: 3 (HKLVHYFNLKINGSDITNTADILLDNYPIMTFDGKDIYPSIAFMVGNKLFLDLYKNIFVE FFRLFRVSVSSQYEELEYYYSCDYTNNRPTIKQHYFYNGEEYTEIDRSKKATNKNSWLIT SGFRLQKWFDSEDCIIYLRSLVRRMEDSNK). In certain aspects, first amino acid sequence is a sequence of OMCP comprising at least 80% identity to SEQ ID NO:1, SEQ ID NO: 2 or SEQ ID NO: 3. For example, the OMCP may have about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identity to SEQ ID NO:1, SEQ ID NO: 2 or SEQ ID NO: 3.

In any of the foregoing embodiments, first amino acid sequence can be a sequence that includes the alphα-helix domains of OMCP or a sequence with at least 80% homology thereto. Thus, in certain aspects, the first amino acid sequence can have at least 80% homology to amino acid positions 48 to 67 and 110 to 147 of SEQ ID NO: 1, amino acid positions 49 to 68 and 111 to 148 of SEQ ID NO: 2, or amino acid positions 48 to 66 and 111 to 148 of SEQ ID NO: 3. For example, the first amino acid sequence can have about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identity to amino acid positions 48 to 67 and 110 to 147 of SEQ ID NO: 1, amino acid positions 49 to 68 and 111 to 148 of SEQ ID NO: 2, or amino acid positions 48 to 66 and 111 to 148 of SEQ ID NO: 3.

### Anti-NKG2D Antibodies

An “anti-NKG2D antibody” means an antibody (as the term is defined herein) that specifically binds an epitope within NKG2D. The term “antibody” includes encompasses a “monoclonal antibody”. “Monoclonal antibody” refers to an antibody that is derived from a single copy or clone, including e.g., any eukaryotic, prokaryotic, or phage clone. Monoclonal antibodies can be produced using e.g., hybridoma techniques well known in the art, as well as recombinant technologies, phage display technologies, synthetic technologies or combinations of such technologies and other technologies readily known in the art. The term “antibody” should also be understood to mean a functional monoclonal antibody, or an immunologically effective fragment thereof; such as an Fab, Fab′, or F(ab′)2 fragment thereof. As long as the protein retains the ability specifically to bind its intended target, it is included within the term “antibody.” Also included within the definition “antibody” for example are single chain forms, generally designated Fv, regions, of antibodies with this specificity. These scFvs are comprised of the heavy and light chain variable regions connected by a linker. Methods of making and using scFvs are known in the art. Additionally, included within the definition “antibody” are single-domain antibodies, generally designated sdAb, which is an antibody fragment consisting of a single monomeric variable antibody domain. A sdAb antibody may be derived from camelids (VHH fragments) or cartilaginous fishes (VNAR fragments). As used herein “humanized antibody” is an antibody that is composed partially or fully of amino acid sequence sequences derived from a human antibody germline by altering the sequence of an antibody having non-human complementarity determining regions (“CDR”). The simplest such alteration may consist simply of substituting the constant region of a human antibody for the murine constant region, thus resulting in a human/murine chimera which may have sufficiently low immunogenicity to be acceptable for pharmaceutical use. Preferably, however, the variable region of the antibody and even the CDR is also humanized by techniques that are by now well known in the art. The framework regions of the variable regions are substituted by the corresponding human framework regions leaving the non-human CDR substantially intact, or even replacing the CDR with sequences derived from a human genome. CDRs may also be randomly mutated such that binding activity and affinity for NKG2D is maintained or enhanced in the context of fully human germline framework regions or framework regions that are substantially human. In certain embodiments, an anti-NKG2D antibody is a Fab, Fab′, or F(ab′)2 fragment.

In any of the foregoing embodiments, where the first amino acid sequence is an anti-NGK2D antibody, the antibody can be, by way of example but not limitation, KYK-1 or KYK-2 as described in Kwong, et al, J Mol Biol. 2008 Dec. 31;384 (5): 1143-56. The light chain of KYK-1 comprises the amino acid sequence set forth in SEQ ID NO: 4 (QPVLTQPSSVSVAPGETARIPCGGDDIETKSVHWYQQKPGQAPVLVIYDDDDRPSGIPE RFFGSNSGNTATLSISRVEAGDEADYYCQVWDDNNDEWVFGGGTQLTVL) and the heavy chain of the KYK-1 comprises the amino acid sequence set forth in SEQ ID NO: 5 (EVQLVESGGGVVQPGGSLRLSCAASGFTFSSYGMHWVRQAPGKGLEWVAFIRYDGSN KYYADSVKGRFTISRDNSKNTLYLQMNSLRAEDTAVYYCAKDRFGYYLDYWGQGTLV TVSS). The light chain of KYK-2 comprises the amino acid sequence set forth in SEQ ID NO: 6 (QSALTQPASVSGSPGQSITISCSGSSSNIGNNAVNWYQQLPGKAPKLLIYYDDLLPS GVSDRFSGSKSGTSAFLAISGLQSEDEADYYCAAWDDSLNGPVFGGGTKLTVL) and the heavy chain of the KYK-2 comprises the amino acid sequence set forth in SEQ ID NO: 7 (QVQLVESGGGLVKPGGSLRLSCAASGFTFSSYGMHWVRQAPGKGLEWVAFIRYDGSN KYYADSVKGRFTISRDNSKNTLYLQMNSLRAEDTAVYYCAKDRGLGDGTYFDYWGQG TTVTVSS).

In another particular embodiment, the anti-NKG2D antibody is an scFv derived from KYK-1. For example, the KYK-1 scFv comprises the amino acid sequence set forth in SEQ ID NO: 8 (QPVLTQPSSVSVAPGETARIPCGGDDIETKSVHWYQQKPGQAPVLVIYDDDDRPSGIPE RFFGSNSGNTATLSISRVEAGDEADYYCQVWDDNNDEWVFGGGTQLTVLGGGGSGGG GSGGGGSEVQLVESGGGVVQPGGSLRLSCAASGFTFSSYGMHWVRQAPGKGLEWVAFI RYDGSNKYYADSVKGRFTISRDNSKNTLYLQMNSLRAEDTAVYYCAKDRFGYYLDYW GQGTLVTVSS). Alternatively, the KYK-1 scFv comprises the amino acid sequence set forth in SEQ ID NO: 9 (EVOLVESGGGVVQPGGSLRLSCAASGFTFSSYGMHWVRQAPGKGLEWVAFIRYDGSN KYYADSVKGRFTISRDNSKNTLYLQMNSLRAEDTAVYYCAKDRFGYYLDYWGQGTLV TVSSGGGGSGGGGSGGGGSQPVLTQPSSVSVAPGETARIPCGGDDIETKSVHWYQQKPG QAPVLVIYDDDDRPSGIPERFFGSNSGNTATLSISRVEAGDEADYYCQVWDDNNDEWVF GGGTQLTVL).

In another particular embodiment, the anti-NKG2D antibody is an scFv derived from KYK-2. For example, the KYK-2 scFv comprises the amino acid sequence set forth in SEQ ID NO: 10 (QSALTQPASVSGSPGQSITISCSGSSSNIGNNAVNWYQQLPGKAPKLLIYYDDLLPSGVS DRFSGSKSGTSAFLAISGLQSEDEADYYCAAWDDSLNGPVFGGGTKLTVLGGGGSGGG GSGGGGSQVQLVESGGGLVKPGGSLRLSCAASGFTFSSYGMHWVRQAPGKGLEWVAFI RYDGSNKYYADSVKGRFTISRDNSKNTLYLQMNSLRAEDTAVYYCAKDRGLGDGTYF DYWGQGTTVTVSS). Alternatively, the KYK-2 scFv comprises the amino acid sequence set forth in SEQ ID NO: 11 (QVQLVESGGGLVKPGGSLRLSCAASGFTFSSYGMHWVRQAPGKGLEWVAFIRYDGSN KYYADSVKGRFTISRDNSKNTLYLQMNSLRAEDTAVYYCAKDRGLGDGTYFDYWGQG TTVTVSSGGGGSGGGGSGGGGSQSALTQPASVSGSPGQSITISCSGSSSNIGNNAVNWYQ QLPGKAPKLLIYYDDLLPSGVSDRFSGSKSGTSAFLAISGLQSEDEADYYCAAWDDSLN GPVFGGGTKLTVL).

As stated above, the various KYK-1 and KYK-2 antibodies or scFv thereof may be combined with any of the cytokines disclosed herein, in the absence or presence of any of the linkers described herein to provide the compositions or chimeric peptides of the present invention. It should also be understood that the KYK-1 and KYK-2 antibodies are examples of antibodies suitable for use in the present compositions and one of skill in the art, based on this disclosure, will understand that other anti-NKG2D antibodies will be suitable as well.

### Tumor Targets

In concept, any cell-surface protein which is sufficiently over-expressed at the cell surface in a given tumor versus the majority of normal tissues would provide a suitable therapeutic target. Non-limiting examples of such targets which are currently used in the clinic are outlined below. In an alternative example, the tumor target might be mutated or be a fusion protein between two naturally occurring proteins, creating unique epitopes which could be targeted. Ideally, binding of these cell surface targets would not inhibit the function of normal tissues. However, it can be appreciated that most proteins expressed by a tumor cell will be expressed to some degree in normal tissues as well.

In any of the embodiments of the present disclosure, the second amino acid sequence of the second domain can be a tumor target binding partner such as, by way of example but not limitation, an antibody variable region or a ligand capable of binding to a peptide (tumor target) on a tumor cell that is either specific to the tumor cell or overexpressed on the tumor cell compared to a non-tumor cell of the same tissue origin as the tumor cell. In certain embodiments, the bispecific or multi-specific construct binds to the tumor target via an antibody variable region. The variable antibody might be expressed as part of a classic antibody backbone, or might be expressed as a linear antibody variable antibody with a short linker between the variable domains. In an alternate embodiment, the construct binds to the tumor target via a naturally occurring ligand to the targeted protein. In some embodiments, the naturally occurring ligand is mutated to either increase or decrease the binding affinity to the targeted protein. Exemplary tumor targets that can be targeted by the polypeptides of the present disclosure are provided in the table below.

| Target | Description |
|---|---|
| ERBB2 | epidermal growth factor receptor 2, receptor tyrosine-protein kinase erbB-2, EGFR2, |
| | HER2, HER-2, p185c-erbB2, NEU, CD340 |
| CD19 | B lymphocyte surface antigen B4, Leu-12 |
| EPCAM | epithelial cell adhesion molecule, tumor-associated calcium signal transducer 1, |
| | TACSTD1, gastrointestinal tumor-associated protein 2, GA733-2, epithelial |
| | glycoprotein 2, EGP-2, KSA, KS1/4 antigen, M4S1, tumor antigen 17-1A, Ep-CAM, |
| | EpCAM, CD326 |
| MS4A1 | membrane-spanning 4-domains subfamily A member 1, CD20 |
| FOLH1 | folate hydrolase, prostate specific membrane antigen, PSMA |
| CEACAM5 | carcinoembryonic antigen-related cell adhesion molecule 5, CEA, CD66e |
| IL3RA | interleukin 3 receptor subunit alpha, ″interleukin 3 receptor, alpha (low affinity |
| PMEL | premelanosome protein, gp100, melanocyte differentiation protein |
| CLEC12A | C-type lectin domain family 12 member A, C-type lectin domain family 12, |
| | member A, CD371, CLL-1, DCAL-2, dendritic cell-associated lectin 2, MICL, |
| | myeloid inhibitory C-type lectin-like receptor |
| KDR | kinase insert domain receptor, vascular endothelial growth factor receptor 2, |
| | VEGFR2, VEGF-R2, FLK1, CD309 |
| EGFR | epidermal growth factor receptor, receptor tyrosine-protein kinase erbB-1, ERBB1, |
| | HER1, HER-1, ERBB |
| TAG-72 | TAG-72, TAG, HMW mucin-like glycoprotein, CA 72-4, tumour associated |
| | glycoprotein 72 |
| GD2 | Disialoganglioside GD2 |
| MICA | MHC Class I Polypeptide-Related Sequence A, PERB11.1, MIC-A, Truncated MHC |
| | Class I Polypeptide-Related Sequence A, MHC Class I Chain-Related Protein A, |
| | Stress Inducible Class I Homolog, MHC Class I Related Sequence A, MHC Class I |
| | Related Chain A, HLA Class I Antigen |
| MICB | MHC Class I Polypeptide-Related Sequence B, PERB11.2, MHC Class I-Like |
| | Molecule PERB11.2-IMX, MHC Class I Chain-Related Protein B, Stress Inducible |
| | Class I Homolog, MHC Class I Mic-B Antigen, MIC-B |
| HLAE | Major Histocompatibility Complex, Class I, E, HLA Class I Histocompatibility |
| | Antigen, Alpha Chain E, MHC Class I Antigen E, HLA-6.2, MHC Class Ib |
| | Antigen, HLA-E, QA1 |
| CD20 | Membrane spanning 4-domains A1, MS4A1, B1, S7, Bp35, CVID5, MS4A2, |
| | LEU-16 |
| CD33 | CD33 molecule, SIGLEC-3, SIGLEC3, p67 |
| CD38 | CD38 molecule, ADPRC 1, ADPRC1 |
| CD123 | Interleukin 3 receptor subunit alapha, IL3R, IL3RAY, IL3RX, IL3RY, hIL-3Ra |
| BCMA | TNF receptor superfamily member 17, BCM, CD269, TNFRSF13A |
| B7H3/CD276 | CD276 molecule, 4Ig-B7-H3, B7-H3, B7RP-2 |
| GPA33 | Glycoprotein A33, A33 |
| SSTR2 | Somatostatin receptor 2 |
| GPC3 | Glypican-3, DGSX, GTR2-2, MXR7, OCI-5, SDYS, SGB, SGBS, SGBS1 |
| CDH3 | Cadherin 3, CDHP, HJMD, PCAD |

In any of the foregoing embodiments, the second domain can be a scFv derived from an antibody. By way of example, but not limitation, the second domain can be an antibody to the epidermal growth factor receptor (EGFR) such as cetuximab. In one embodiment, the variable light chain of cetuximab comprises the amino acid sequence set forth in SEQ ID NO: 12 (DILLTQSPVILSVSPGERVSFSCRASQSIGTNIHWYQQRINGSPRLLIKYASESISGIPSRFS GSGSGTDFTLSINSVESEDIADYYCQQNNNWPTTFGAGTKLELKRTVA) and the variable heavy chain of cetuximab comprise the amino acid sequence set forth in SEQ ID NO: 13 (QVQLKQSGPGLVQPSQSLSITCTVSGFSLTNYGVHWVRQSPGKGLEWLGVIWSGGNTD YNTPFTSRLSINKDNSKSQVFFKMNSLQSNDTAIYYCARALTYYDYEFAYWGQGTLVT VSA). In one embodiment, the fusion proteins of any embodiments of the present invention comprise a cetuximab scFv comprising the amino acid sequence set forth in SEQ ID NO: 14 (DILLTQSPVILSVSPGERVSFSCRASQSIGTNIHWYQQRTNGSPRLLIKYASESISGIPSRFS GSGSGTDFTLSINSVESEDIADYYCQQNNNWPTTFGAGTKLELKRTVAGGGGSGGGGSG GGGSQVQLKQSGPGLVQPSQSLSITCTVSGFSLTNYGVHWVRQSPGKGLEWLGVIWSG GNTDYNTPFTSRLSINKDNSKSQVFFKMNSLQSNDTAIYYCARALTYYDYEFAYWGQG TLVTVSA) which includes a GGGS (SEQ ID NO: 37) linker between the variable light and heavy chains. In certain aspects, the variable light chain of the scFV can include a sequence with at least about 80% homology to SEQ ID NO: 12. For example, the VL may have about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identity to SEQ ID NO: 12. In certain aspects, the variable heavy chain of the scFv can include a sequence with at least about 80% homology to SEQ ID NO: 13. For example, the VH may have about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identity to SEQ ID NO: 13. In certain aspects, the scFV can include a sequence with at least about 80% homology to SEQ ID NO: 14. For example, the scFv may have about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identity to SEQ ID NO: 14.

Thus, in one embodiment, the fusion proteins of any of the relevant embodiments of the present invention may comprise OMCP and cetuximab scFV. One exemplary embodiment of this fusion protein comprises the amino acid sequence set forth in SEQ ID NO: 15

(DILLTQSPVILSVSPGERVSFSCRASQSIGTNIHWYQQRTNGSPRLLIKYASESISGIPSRF
SGSGSGTDFTLSINSVESEDIADYYCQQNNNWPTTFGAGTKLELKRTVAGGGGSGGGGSG
GGGSQVQLKQSGPGLVQPSQSLSITCTVSGFSLTNYGVHWVRQSPGKGLEWLGVIWSG
GNTDYNTPFTSRLSINKDNSKSQVFFKMNSLQSNDTAIYYCARALTYYDYEFAYWGQG
TLVTVSAGGGGSGGGGSGGGGSHKLAFNFNLEINGSDTHSTVDVYLDDSQIITFDGKDIR
PTIPFMIGDEIFLPFYKNVFSEFFSLFRRVPTSTPYEDLTYFYECDYTDNKSTFDQFYLYNG
EEYTVKTQEATNKNMWLTTSEFRLKKWFDGEDCIMHLRSLVRKMEDSKRNTG).

### Tri-Specific Fusion Proteins

The therapeutic fusion peptides of the present disclosure can include tri-and quad-specific therapeutics. A tri-specific configuration can include antibody variable regions against two separate tumor targets in addition to the first domain. Cancer cells are known to have variable protein expression patterns even within the same tumor. Therefore, incorporation of multiple tumor-targeted ligands would increase the likelihood of a given surface receptor being expressed on every cell. As a non-limiting example, variable regions against both ERBB2 and EGFR could be included. Antibody variable regions could be incorporated which are specific against any combination of tumor-specific targets including ERBB2, CD19, EPCAM, MS4A1, FOLH1, CEACAM5, IL3RA, PMEL, CLEC12A, KDR, EGFR, TAG-72 (tumor associated glycoprotein 72), CD20, CD123, CD33, BCMA, CD38, B7H3/CD276, GPA33, SSTR2, GPC3, and CDH3.

A tri-specific configuration might also include two NKG2D binding moieties, such as NKG2D ligand or aNKG2D antibody variable regions. In such an embodiment, the binding of multiple NKG2D receptors at the surface of the protein might contribute to NKG2D dimerization and NKG2D pathway activation, resulting in activation of the cytotoxic NK and CD8⁺ T cells. In an alternate configuration, the functional activation of NKG2D could be optimized via the insertion of one or more NKG2D ligands sequentially with a short linker sequence between each NKG2D ligand. In a specific embodiment of this configuration, the NKG2D ligands can be OMCP or a variant thereof as disclosed herein.

In any of the foregoing embodiments, a polypeptide of the present disclosure can further include, in addition to the first domain and second domain as described, a third domain. In certain aspects, the third domain comprises a third amino acid sequence that is capable of binding to a peptide on the tumor cell, where the peptide is either specific to the tumor cell or overexpressed on the tumor cell compared to a non-tumor cell of the same tissue origin as the tumor cell. In certain aspects, the third amino sequence is capable of binding to the same peptide that the second amino acid sequence is capable of binding to. In certain aspects, the third amino acid sequence is capable of binding to a different peptide than the second amino acid sequence is capable of binding to. It should be understood that the foregoing description of embodiments with respect to the second domain applies to the third domain when it includes a third amino acid sequence that is capable of binding to a peptide on the tumor cell.

In certain aspects, the third domain comprises a third amino acid sequence that is capable of binding to human NKG2D. It should be understood that the description of embodiments with respect to the first domain applies to the third domain when it includes a third amino acid sequence that is capable of binding to human NKG2D.

In certain aspects, the third domain comprises a Fc antibody domain. In certain aspects the Fc antibody domain can comprise a mutation that prevents the Fc antibody domain from binding to CD16.

In certain aspects, the third domain comprises a cytokine.

In certain aspects, the third domain can be a linker. In certain aspects, where the third domain is a linker, the linker can be positioned between the first domain and the second domain.

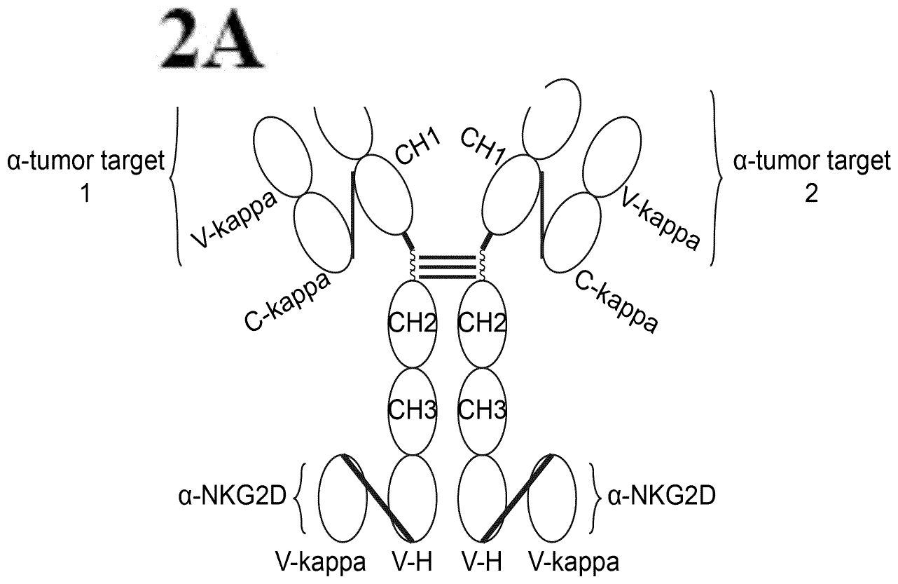

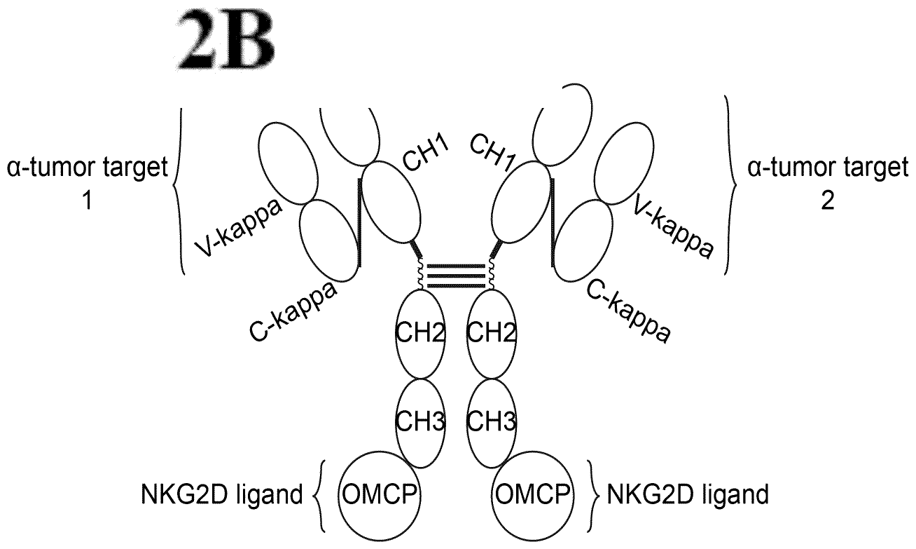

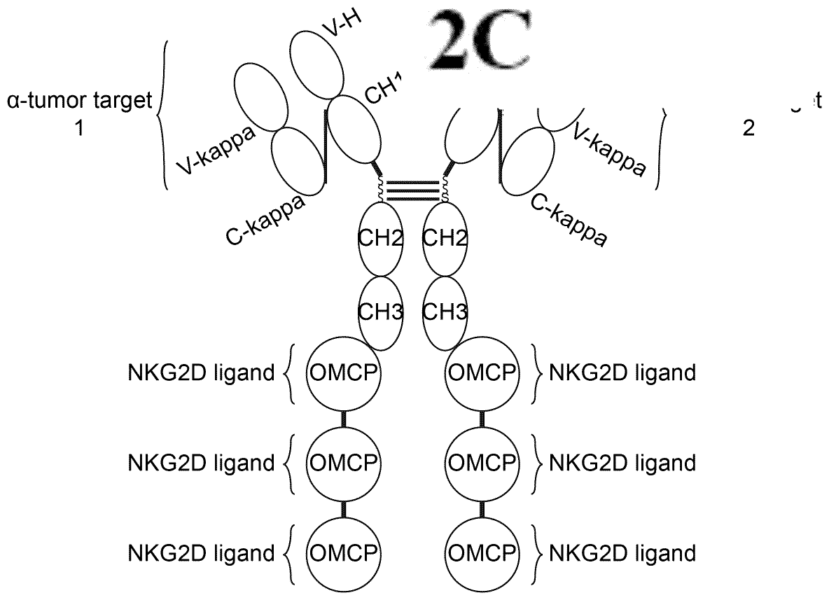

Exemplary tri-specific polypeptides are shown in FIGS. 2A-2C.

### Quad-Specific Fusion Proteins

In an alternate configuration, the fusion protein can incorporate multiple lymphocyte targeting proteins as well as multiple tumor targeting proteins, creating a quad-specific protein. In one example, both lymphocyte targeting ligands would bind NKG2D, and specifically be OMCP. In an alternate example, two separate lymphocyte surface receptors would be selected. This would enable the engagement of lymphocytes in various activation states, or alternatively might bias therapeutic activation more heavily towards either NK cell activation or CD8⁺ T cell activation. In a non-limiting example, the two lymphocyte ligands could be selected from any combination of either anti-NKG2D antibody, OMCP, anti-CD3e (biased to CD8⁺ T cells), or anti-FCGR1 (biased to NK cells). Antibody variable regions could be incorporated which are specific against any combination of tumor-specific targets including ERBB2, CD19, EPCAM, MS4A1, FOLH1, CEACAM5, IL3RA, PMEL, CLEC12A, KDR, EGFR, TAG-72 (tumor associated glycoprotein 72), CD20, CD123, CD33, BCMA, CD38, B7H3/CD276, GPA33, SSTR2, GPC3, and CDH3.

In any of the foregoing embodiments, where the polypeptide of the present disclosure includes three domains, the polypeptide can include a fourth domain that includes a fourth amino acid sequence. In certain aspects, fourth amino acid sequence is capable of binding to a peptide on the tumor cell, where the peptide is either specific to the tumor cell or overexpressed on the tumor cell compared to a non-tumor cell of the same tissue origin as the tumor cell. In certain aspects, the fourth amino sequence is capable of binding to the same peptide that the second or third amino acid sequence is capable of binding to. In certain aspects, the fourth amino acid sequence is capable of binding to a different peptide than the second and third amino acid sequences are capable of binding to. It should be understood that the foregoing description of embodiments with respect to the second domain applies to the fourth domain when it includes a fourth amino acid sequence that is capable of binding to a peptide on the tumor cell.

In certain aspects, the fourth amino acid sequence that is capable of binding to human NKG2D. It should be understood that the description of embodiments with respect to the first domain applies to the fourth domain when it includes a third amino acid sequence that is capable of binding to human NKG2D.

In certain aspects, the fourth domain comprises a Fc antibody domain. In certain aspects the Fc antibody domain can comprise a mutation that prevents the Fc antibody domain from binding to CD16.

In certain aspects, the fourth domain comprises a cytokine.

In certain aspects, the fourth domain can be a linker.

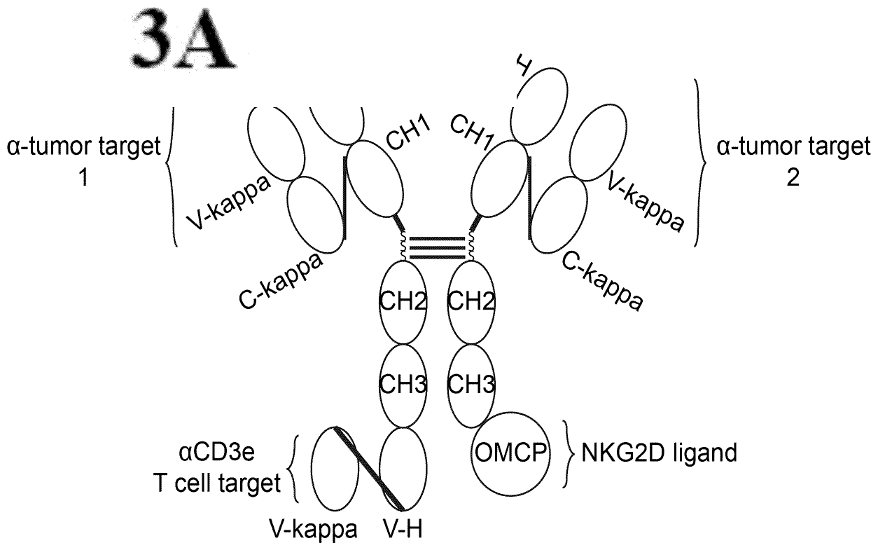

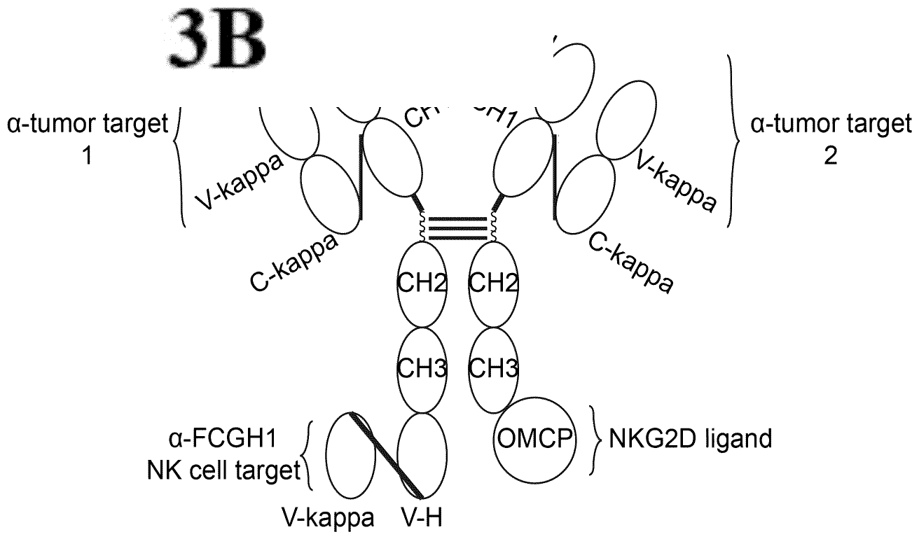

Exemplary quad-specific polypeptides of the present disclosure are shown in FIGS. 3A-3B.

It should be understood that a polypeptide of the present disclosure can include more than four specific domains and can include combinations of the domains disclosed herein in addition to the first domain and the second domain.

### Fc Antibody Domains

In any of the embodiments of the present disclosure, a polypeptide can include a Fc antibody domain from an antibody. In some embodiments, the Fc antibody domain can include a Hinge portion, a CH3 portion and a CH2 portion. By way of example, but not limitation, the Fc antibody domain can include the sequence of SEQ ID NO: 21

(EPKSCDKTHTCPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKALPAPI
EKTISKAKGQPREPQVYTLPPSREEMTKNQVSLTCLVKGFYPSDIAVEWESNGQPENNY
KTTPPVLDSDGSFFLYSKLTVDKSRWQQGNVFSCSVMHEALHNHYTQKSLSLSPGK)

which is a wild-type sequence for IgG1 Fc.

In certain aspects, the Fc antibody domain can further include domains that promote heterodimerization. In some embodiments, the Fc portion can include a knob domain and a hole domain that allow for heterodimerization of the two chains. By way of example, but not limitation, the Fc antibody domain can include a knob domain and a hole domain such as the knob domain of SEQ ID NO: 22

(EPKSCDKTHTCPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKALPAPI
EKTISKAKGQPREPQVYTLPPSREEMTKNQVSLYCLVKGFYPSDIAVEWESNGQPENNY
KTTPPVLDSDGSFFLYSKLTVDKSRWQQGNVFSCSVMHEALHNHYTQKSLSLSPGK)

and the hole domain of SEQ ID NO: 23

(EPKSCDKTHTCPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKALPAPI
EKTISKAKGQPREPQVYTLPPSREEMTKNQVSLTCLVKGFYPSDIAVEWESNGQPENNY
KTTPPVLDSDGSFFLTSKLTVDKSRWQQGNVFSCSVMHEALHNHYTQKSLSLSPGK).

By way of further example, but not limitation, the knob domain can include the sequence of SEQ ID NO: 24

(EPKSCDKTHTCPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKALPAPI
EKTISKAKGQPREPQVYTLPPSREEMTKNQVSLYCLVKGFYPSDIAVEWESNGQPENNY
KTTPPVLDSDGSFALYSKLTVDKSRWQQGNVFSCSVMHEALHNHYTQKSLSLSPGK)

and the hole domain can include the sequence of SEQ ID NO: 25

(EPKSCDKTHTCPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKALPAPI
EKTISKAKGQPREPQVYTLPPSREEMTKNQVSLTCLVKGFYPSDIAVEWESNGQPENNY
KTWPPVLDSDGSFFLTSKLTVDKSRWQQGNVFSCSVMHEALHNHYTQKSLSLSPGK).

By way of even further example, but not limitation, the knob domain can include the sequence of SEQ ID NO: 26

(EPKSCDKTHTCPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKALPAPI
EKTISKAKGQPREPQVYTLPPSREEMTKNQVSLWCLVKGFYPSDIAVEWESNGQPENNY
KTTPPVLDSDGSFFLYSKLTVDKSRWQQGNVFSCSVMHEALHNHYTQKSLSLSPGK)

and the hole domain can include the sequence of SEQ ID NO: 27

(EPKSCDKTHTCPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKALPAPI
EKTISKAKGQPREPQVYTLPPSREEMTKNQVSLSCAVKGFYPSDIAVEWESNGQPENNY
KTTPPVLDSDGSFFLVSFLTVDKSRWQQGNVFSCSVMHEALHNHYTQKSLSLSPGK)

which provides a phage-display knob-in-hole system for generating heterodimers.

In some embodiments, the Fc antibody domain can include domains for SEED heterodimerization. By way of example, bot limitation, the Fc antibody domain can include a domain having the sequence of SEQ ID NO: 28

(EPKSSDKTHTCPPCPAPELLGGPSVFLFPPKPKDTLMISRTPEVTCWVDVSHEDPEVKFN
WYVDGVEVHNAKTKPREEQYNSTYRVVSVLTVLHQDWLNGKEYKCKVSNKALPAPIE
KTISKAKGQPFRPEVHLLPPSREEMTKNQVSLTCLARGFYPKDIAVEWESNGQPENNYK
TTPSRQEPSQGTTTFAVTSKLTVDKSRWQQGNVFSCSVMHEALHNHYTQKTISLSPGK)

and a domain having the sequence of SEQ ID NO: 29

(EPKSSDKTHTCPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKALPAPI
EKTISKAKGQPREPQVYTLPPPSEELALNELVTLTCLVKGFYPSDIAVEWLQGSQELPRE
KYLTWAPVLDSDGSFFLYSILRVAAEDWKKGDTFSCSVMHEALHNHYTQKSLDRSPGK)

which preferentially form heterodimers.

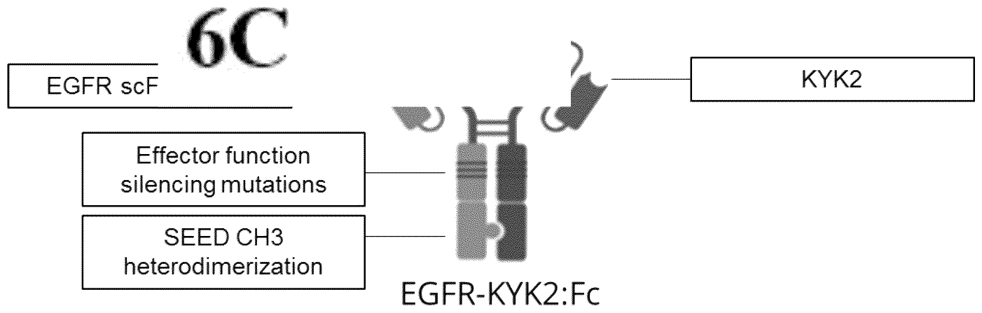

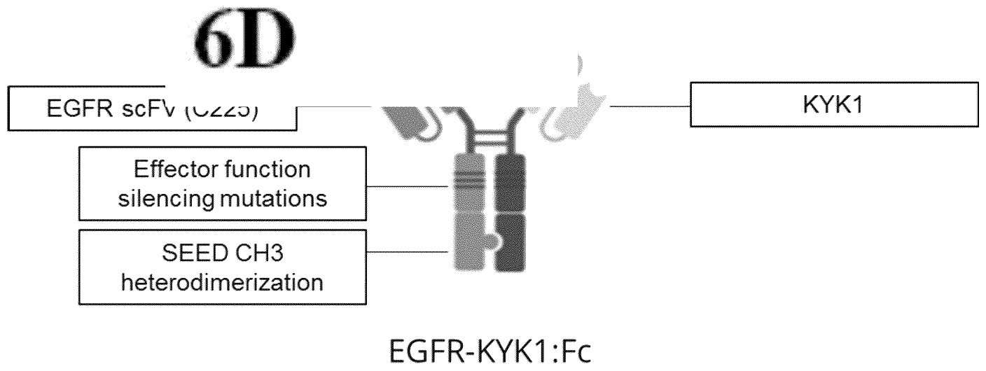

By preferentially forming heterodimers, the polypeptide with the Fc antibody domain of the present disclosure can have two different binding moieties, one attached to each portion of the Fc antibody domain. For example, as shown in FIGS. 6A-6D, exemplary polypeptides of the present disclosure can include two specific binding moieties combined with an Fc antibody domain which, in the case of FIGS. 6A-6D includes a SEED heterodimerization design.

In some embodiments, the Fc antibody can further include a mutation or mutations that prevent binding of the Fc portion to CD16. FIGS. 6A-6B further exemplify such designs which include effector function silencing mutations. By way of example, but not limitation, effector silencing mutations include L234A, L235P, P329G and combinations thereof relative to native human IgG1. In SEQ ID NO: 21, these mutations would occur at positions 19, 20 and 114of SEQ ID NO: 21 (L19, L20 and P114). Thus, effector function silencing mutations can be at a corresponding position in the Fc antibody domain of the polypeptides of the present disclosure, to the extent that corresponding positions are present.

In some embodiments the Fc antibody domain can further include a mutation or mutations that enable purification of the construct. By way of example, but not limitation, such mutations can include T307P, L309Q, Q311R and combinations thereof relative to native human IgG1. In SEQ ID NO: 21, these mutations would occur at positions 19, 20 and 114 of SEQ ID NO: 21 (T92, L94 and Q96).

In some embodiments, a polypeptide of the present disclosure can include a monomeric Fc antibody domain. A monomeric Fc domain can be used to increase the half-life of the polypeptides of the present disclosure. By way of example, but not limitation, the monomeric Fc domain can include the sequence of SEQ ID NO: 30

(EPKSCDKTHTCPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKALPAPI
EKTISKAKGQPREPQVYTSPPSRDELTKNQVSLRCHVKGFYPSDIAVEWESNGQPENNY
KTTKPVLDSDGSFFLYSKLTVDKSRWQQGNVFSCSVMHEALHNHYTQKSLSLSPGK)

which is a monomeric Fc domain with L351S, T366R, L368H and P396K mutations (corresponding to positions 136, 151, 153 and 181 in SEQ ID NO: 30, respectively).

It should be understood that a Fc antibody domain in the polypeptides of the present disclosure can vary from the foregoing exemplary embodiments due to mutations, additions, deletions and other modifications to the Fc antibody domain. By way of example but not limitation, a Fc antibody domain can include an amino acid sequence with at least 80%, 81%, 82%, 83%, 84%, 85%, 86%, 87%, 88%, 89%, 90%, 91%, 92%, 93%, 94%, 95%, 96%, 97%, 98%, 99% or 100% homology to any of SEQ ID NOs: 21-30.

### Linkers

It should be understood that the polypeptides of the present disclosure can include linkers, as disclosed herein, and between domains of the polypeptides. In some embodiments, the linker can include (GGGGS)ₙ where n is an integer of at least 1. By way of example, but not limitation, n can be 1, 2, 3, 4, 5 or more. Linkers are well known to those of skill in the art and any suitable linker can be used.

### Cytokines

A “cytokine” is a small protein (˜5-20 kDa) that is important in cell signaling. Cytokines are released by cells and affect the behavior of other cells and/or the cells that release the cytokine. Non-limiting examples of cytokines include chemokines, interferons, interleukins, lymphokines, tumor necrosis factor, monokines, and colony stimulating factors. Cytokines may be produced by a broad range of cells including, but not limited to, immune cells such as macrophages, B lymphocytes, T lymphocytes, mast cells and monocytes, endothelial cells, fibroblasts and stromal cells. A cytokine may be produced by more than one type of cell. Cytokines act through receptors and are especially important in the immune system, modulate the balance between humoral and cell-based immune responses, and regulate maturation, growth and responsiveness of cell populations. Cytokines are important in host responses to infection, immune responses, inflammation, trauma, sepsis, cancer and reproduction. A cytokine of the invention may be a naturally occurring cytokine or may be a mutated version of a naturally occurring cytokine. As used herein, “naturally occurring”, which may also be referred to as wild-type, includes allelic variances. A mutated version or “mutant” of a naturally occurring cytokine refers to specific mutations that have been made to the naturally occurring sequence to alter the function, activity and/or specificity of the cytokine. In one embodiment, the mutations may enhance the function, activity and/or specificity of the cytokine. In another embodiment, the mutations may decrease the function, activity and/or specificity of the cytokine. The mutation may include deletions or additions of one or more amino acid residues of the cytokine.

Cytokines may be classified based on structure. For example, cytokines may be classified into four types: the four-α-helix bundle family, the IL1 family, the IL 17 family and the cysteine-knot cytokines. Members of the four-α-helix bundle family have three-dimensional structures with four bundles of α-helices. This family is further divided into three sub-families: the IL2 subfamily, the interferon (IFN) subfamily and the IL10 subfamily. The IL2 subfamily is the largest and comprises several non-immunological cytokines including, but not limited to, erythropoietin (EPO) and thrombopoietin (TPO).

In any of the foregoing embodiments, the cytokine can be a cytokine from the four-α-helix bundle family or a mutant thereof. A skilled artisan would be able to determine cytokines within the four-α-helix bundle family.

In any of the foregoing embodiments, the cytokine can be an IL2 subfamily cytokine or a mutant thereof. Non-limiting examples of members of the IL2 subfamily include IL2, IL4, IL7, IL9, IL15 and IL21. In a specific embodiment, the cytokine is IL2 or a mutant thereof. In any of the foregoing embodiments, the cytokine can be IL15 or a mutant thereof. The sequence information for the full length human IL15 amino acid sequence can be found using, for example, the GenBank accession number CAG46777.1, AAI00962.1 or AAI00963.1. The sequence information for the full length human IL15 mRNA sequence can be found using, for example, the GenBank accession number CR542007.1, KJ891469.1, NM_172175.2, NM_000585.4 or CR541980.1. A skilled artisan will appreciate that IL 15 may be found in a variety of species and methods of identifying analogs or homologs of IL15 are known in the art as described in detail below.

In any of the foregoing embodiments, the cytokine can be an IL1 family cytokine or a mutant thereof. The IL1 family is a group of 11 cytokines, which plays a central role in the regulation of immune and inflammatory responses. Generally, the IL1 family of cytokines are proinflammatory cytokines that regulate and initiate inflammatory responses. Non-limiting examples of IL1 family cytokines include IL1α, IL1β, ILIRa, IL18, IL-36Ra, IL36α, IL37,IL36β, IL36γ, IL3δ, and IL33. IL1 family members have a similar gene structure. A skilled artisan would be able to determine cytokines within the IL1 family.

In any of the foregoing embodiments, the cytokine can be IL18 or a mutant thereof. The sequence information for the full length human IL18 amino acid sequence can be found using, for example, the GenBank accession number CAG46771.1. The sequence information for the full length human IL18 mRNA sequence can be found using, for example, the GenBank accession number KR710147.1, CR542001.1, CR541973.1 or KJ897054.1. A skilled artisan will appreciate that IL18 may be found in a variety of species and methods of identifying analogs or homologs of IL18 are known in the art.

In any of the foregoing embodiment, the cytokine can be an interferon subfamily cytokine or a mutant thereof. Interferons are named for their ability to “interfere” with viral replication by protecting cells from virus infection. IFNs also have other functions: they activate immune cells, such as natural killer cells and macrophages; they increase host defenses by up-regulating antigen presentation by virtue of increasing the expression of major histocompatibility complex (MHC) antigens. Based on the type of receptor through which they signal, human interferons have been classified into three major types: Type I IFN, Type II IFN, and Type III IFN. Type I IFNs bind to a specific cell surface receptor complex known as the IFN-α/β receptor (IFNAR) that consists of IFNAR1 and IFNAR2 chains. Non-limiting examples of type I interferons present in humans are IFN-α, IFN-β, IFN-ε, IFN-κ and IFN-ω. Thus, in certain embodiments, a cytokine of the composition is a Type 1 IFN cytokine or a mutant thereof, including, but not limited to wild-type and mutant forms of IFN-α, IFN-β, IFN-ε, IFN-κ and IFN-ω. Type II IFNs bind to IFNGR that consists of IFNGR1 and IFNGR2 chains. Non-limiting examples of type II interferons present in humans is IFN-γ. Thus, in certain embodiments, a cytokine of the composition is a Type II IFN cytokine or a mutant thereof, including, but not limited to wild-type and mutant forms of IFN-γ. Type III IFNs signal through a receptor complex consisting of IL10R2 (also called CRF2-4) and IFNLR1 (also called CRF2-12). Non-limiting examples of type III interferons include IFN-λ1, IFN-λ2 and IFN-λ3 (also called ILλ9, ILλ8A and ILλ8B respectively). Thus, in certain embodiments, a cytokine of the composition is a Type III IFN cytokine or a mutant thereof, including, but not limited to wild-type and mutant forms of IFN-λ1, IFN-λ2 and IFN-λ3.

In any of the foregoing embodiments, the cytokine can be a member of the tumor necrosis factor superfamily (TNSF), or a mutant thereof. TNSF members are pro-inflammatory cytokines mainly expressed by immune cells which induce an inflammatory state and stimulate immune cell function. At least 18 TNSF homologues exist, including but not limited to, TNF (TNFalpha), CD40L (TNFSF5), CD70 (TNFSF7), EDA, FASLG (TNFSF6), LTA (TNFSF1), LTB (TNFSF3), TNFSF4 (OX40L), TNFSF8 (CD153), TNFSF9 (4-1BBL), TNFSF10 (TRAIL), TNFSF11 (RANKL), TNFSF12 (TWEAK), TNFSF13, TNFSF13B, TNFSF14, TNFSF15,TNFSF18. Thus, in certain embodiments, a cytokine of the composition is a member of the tumor necrosis factor superfamily or a mutant thereof, including, but not limited to TNF (TNFalpha), CD40L (TNFSF5), CD70 (TNFSF7), EDA, FASLG (TNFSF6), LTA (TNFSF1), LTB (TNFSF3), TNFSF4 (OX40L), TNFSF8 (CD153), TNFSF9 (4-1BBL), TNFSF10 (TRAIL), TNFSF11 (RANKL), TNFSF12 (TWEAK), TNFSF13, TNFSF13B, TNFSF14, TNFSF15,TNFSF18.

In another configuration, an immune-modulatory cytokine could be incorporated into the fusion protein design. For example, a cytokine could be incorporated within the linker between the lymphocyte-specific ligand and the tumor-targeted antibody. In another example, a cytokine could be incorporated from one chain of a fusion protein containing an Fc heavy chain. In a non-limiting example, the active molecule could be selected from either a naturally occurring form or mutated form of IL1, IL2, IL7, IL12, IL15, IL18, IL21, TNFα, IFNα, IFNλ.

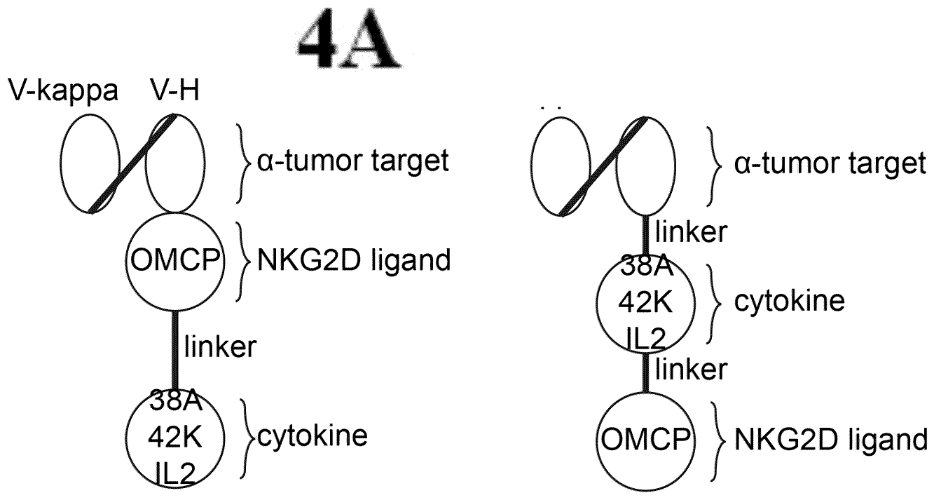

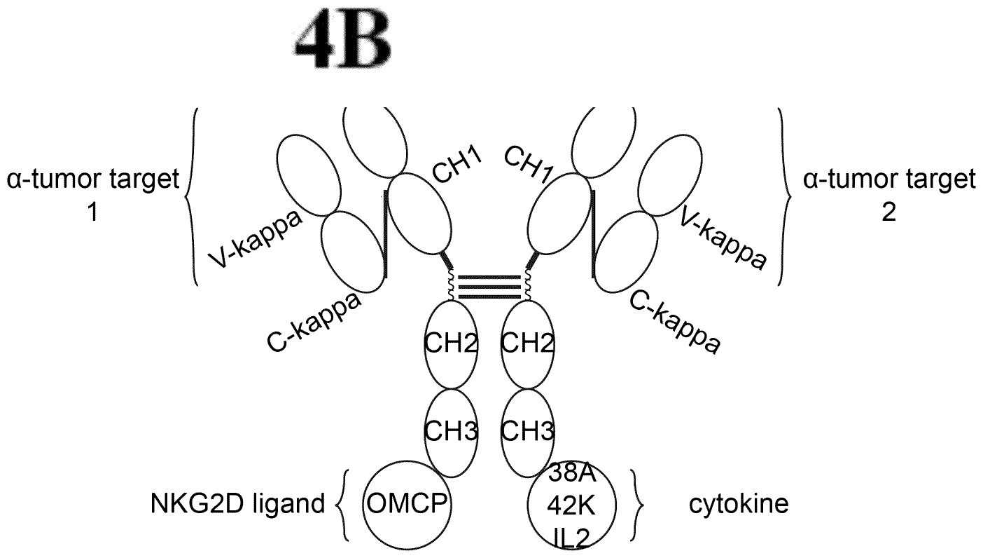

Exemplary polypeptides of the present disclosure that include a cytokine are shown in FIGS. 4A-4B.

PEGylation and glycosylation

In certain embodiments wherein the NKG2D ligand is OMCP or a variant thereof, OMCP or the variant thereof can be modified for improved systemic half-life and reduced dosage frequency. In any of the foregoing embodiments where the polypeptide include OMCP or a variant thereof, N-glycans may be added to OMCP or variant thereof. While the biological function is typically determined by the protein component, carbohydrate can play a role in molecular stability, solubility, in vivo activity, serum half-life, and immunogenicity. The sialic acid component of carbohydrate in particular, can extend the serum half-life of protein therapeutics. Accordingly, new N-linked glycosylation consensus sequences may be introduced into desirable positions in the peptide backbone to generate proteins with increased sialic acid containing carbohydrate, thereby increasing in vivo activity due to a longer serum half-life. In another embodiment, PEG may be added to OMCP or the variant thereof. Methods of conjugating PEG to a protein are standard in the art. For example, see Kolate et al, Journal of Controlled Release 2014; 192 (28): 67-81, which is hereby incorporated by reference in its entirety. In any of the foregoing embodiments, a polypeptide of the present disclosure may comprise OMCP or a variant thereof comprising PEG and/or one or more N-glycans. In any of the foregoing embodiments, PEG is selected from the group consisting of PEG-10K, PEG-20K and PEG-40K.

### De-Immunization

Still further, the fusion protein of the disclosure may be modified to remove T cell epitopes. T cell epitopes can stimulate an immunogenic reaction upon administration of a composition to a subject. Through their presentation to T cells, they activate the process of anti-drug antibody development. Preclinical screening for T cell epitopes may be performed in silico, followed by in vitro and in vivo validation. T cell epitope-mapping tools such as EpiMatrix can be highly accurate predictors of immune response. Deliberate removal of T cell epitopes may reduce immunogenicity.

### Pharmaceutical Compositions

The present disclosure also provides pharmaceutical compositions. The pharmaceutical composition may comprise one of the therapeutic fusion proteins described herein as an active ingredient and at least one pharmaceutically acceptable excipient.

The pharmaceutically acceptable excipient may be a diluent, a binder, a filler, a buffering agent, a pH modifying agent, a disintegrant, a dispersant, a preservative, a lubricant, taste-masking agent, a flavoring agent, or a coloring agent. The amount and types of excipients utilized to form pharmaceutical compositions may be selected according to known principles of pharmaceutical science.

In one embodiment, the excipient may be a diluent. The diluent may be compressible (i.e., plastically deformable) or abrasively brittle. Non-limiting examples of suitable compressible diluents include microcrystalline cellulose (MCC), cellulose derivatives, cellulose powder, cellulose esters (i.e., acetate and butyrate mixed esters), ethyl cellulose, methyl cellulose, hydroxypropyl cellulose, hydroxypropyl methylcellulose, sodium carboxymethylcellulose, corn starch, phosphated corn starch, pregelatinized corn starch, rice starch, potato starch, tapioca starch, starch-lactose, starch-calcium carbonate, sodium starch glycolate, glucose, fructose, lactose, lactose monohydrate, sucrose, xylose, lactitol, mannitol, malitol, sorbitol, xylitol, maltodextrin, and trehalose. Non-limiting examples of suitable abrasively brittle diluents include dibasic calcium phosphate (anhydrous or dihydrate), calcium phosphate tribasic, calcium carbonate, and magnesium carbonate.

In another embodiment, the excipient may be a binder. Suitable binders include, but are not limited to, starches, pregelatinized starches, gelatin, polyvinylpyrrolidone, cellulose, methylcellulose, sodium carboxymethylcellulose, ethylcellulose, polyacrylamides, polyvinyloxoazolidone, polyvinylalcohols, C12-C18 fatty acid alcohol, polyethylene glycol, polyols, saccharides, oligosaccharides, polypeptides, oligopeptides, and combinations thereof.

In another embodiment, the excipient may be a filler. Suitable fillers include, but are not limited to, carbohydrates, inorganic compounds, and polyvinylpyrrolidone. By way of non-limiting example, the filler may be calcium sulfate, both di-and tri-basic, starch, calcium carbonate, magnesium carbonate, microcrystalline cellulose, dibasic calcium phosphate, magnesium carbonate, magnesium oxide, calcium silicate, talc, modified starches, lactose, sucrose, mannitol, or sorbitol.

In still another embodiment, the excipient may be a buffering agent. Representative examples of suitable buffering agents include, but are not limited to, phosphates, carbonates, citrates, tris buffers, and buffered saline salts (e.g., Tris buffered saline or phosphate buffered saline).

In various embodiments, the excipient may be a pH modifier. By way of non-limiting example, the pH modifying agent may be sodium carbonate, sodium bicarbonate, sodium citrate, citric acid, or phosphoric acid.

In a further embodiment, the excipient may be a disintegrant. The disintegrant may be non-effervescent or effervescent. Suitable examples of non-effervescent disintegrants include, but are not limited to, starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pecitin, and tragacanth. Non-limiting examples of suitable effervescent disintegrants include sodium bicarbonate in combination with citric acid and sodium bicarbonate in combination with tartaric acid.

In yet another embodiment, the excipient may be a dispersant or dispersing enhancing agent. Suitable dispersants may include, but are not limited to, starch, alginic acid, polyvinylpyrrolidones, guar gum, kaolin, bentonite, purified wood cellulose, sodium starch glycolate, isoamorphous silicate, and microcrystalline cellulose.

In another alternate embodiment, the excipient may be a preservative. Non-limiting examples of suitable preservatives include antioxidants, such as BHA, BHT, vitamin A, vitamin C, vitamin E, or retinyl palmitate, citric acid, sodium citrate; chelators such as EDTA or EGTA; and antimicrobials, such as parabens, chlorobutanol, or phenol.

In a further embodiment, the excipient may be a lubricant. Non-limiting examples of suitable lubricants include minerals such as talc or silica; and fats such as vegetable stearin, magnesium stearate or stearic acid.

The weight fraction of the excipient or combination of excipients in the composition may be about 99% or less, about 97% or less, about 95% or less, about 90% or less, about 85% or less, about 80% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, about 5% or less, about 2%, or about 1% or less of the total weight of the composition.

The composition can be formulated into various dosage forms and administered by a number of different means that will deliver a therapeutically effective amount of the active ingredient. Such compositions can be administered orally, parenterally, or topically in dosage unit formulations containing conventional nontoxic pharmaceutically acceptable carriers, adjuvants, and vehicles as desired. Topical administration may also involve the use of transdermal administration such as transdermal patches or iontophoresis devices. The term “parenteral” as used herein includes subcutaneous, intravenous, intramuscular, or intrasternal injection, or infusion techniques. Formulation of drugs is discussed in, for example, Gennaro, A. R., Remington's Pharmaceutical Sciences, Mack Publishing Co., Easton, Pa. (18th ed, 1995), and Liberman, H. A. and Lachman, L., Eds., Pharmaceutical Dosage Forms, Marcel Dekker Inc., New York, N.Y. (1980).

Solid dosage forms for oral administration include capsules, tablets, caplets, pills, powders, pellets, and granules. In such solid dosage forms, the active ingredient is ordinarily combined with one or more pharmaceutically acceptable excipients, examples of which are detailed above. Oral preparations may also be administered as aqueous suspensions, elixirs, or syrups. For these, the active ingredient may be combined with various sweetening or flavoring agents, coloring agents, and, if so desired, emulsifying and/or suspending agents, as well as diluents such as water, ethanol, glycerin, and combinations thereof.

For parenteral administration (including subcutaneous, intradermal, intravenous, intramuscular, and intraperitoneal), the preparation may be an aqueous or an oil-based solution. Aqueous solutions may include a sterile diluent such as water, saline solution, a pharmaceutically acceptable polyol such as glycerol, propylene glycol, or other synthetic solvents; an antibacterial and/or antifungal agent such as benzyl alcohol, methyl paraben, chlorobutanol, phenol, thimerosal, and the like; an antioxidant such as ascorbic acid or sodium bisulfite; a chelating agent such as etheylenediaminetetraacetic acid; a buffer such as acetate, citrate, or phosphate; and/or an agent for the adjustment of tonicity such as sodium chloride, dextrose, or a polyalcohol such as mannitol or sorbitol. The pH of the aqueous solution may be adjusted with acids or bases such as hydrochloric acid or sodium hydroxide. Oil-based solutions or suspensions may further comprise sesame, peanut, olive oil, or mineral oil.

In certain embodiments, a composition comprising a therapeutic fusion peptide of the present disclosure can be encapsulated in a suitable vehicle to either aid in the delivery of the compound to target cells, to increase the stability of the composition, or to minimize potential toxicity of the composition. As will be appreciated by a skilled artisan, a variety of vehicles are suitable for delivering a composition of the present invention. Non-limiting examples of suitable structured fluid delivery systems may include nanoparticles, liposomes, microemulsions, micelles, dendrimers and other phospholipid-containing systems. Methods of incorporating compositions into delivery vehicles are known in the art.

In one alternative embodiment, a liposome delivery vehicle may be utilized. Liposomes, depending upon the embodiment, are suitable for delivery of peptides in view of their structural and chemical properties. Generally speaking, liposomes are spherical vesicles with a phospholipid bilayer membrane. The lipid bilayer of a liposome may fuse with other bilayers (e.g., the cell membrane), thus delivering the contents of the liposome to cells. In this manner, the compound of the invention may be selectively delivered to a cell by encapsulation in a liposome that fuses with the targeted cell's membrane.

Liposomes may be comprised of a variety of different types of phospholipids having varying hydrocarbon chain lengths. Phospholipids generally comprise two fatty acids linked through glycerol phosphate to one of a variety of polar groups. Suitable phospholipids include phosphatidic acid (PA), phosphatidylserine (PS), phosphatidylinositol (PI), phosphatidylglycerol (PG), diphosphatidylglycerol (DPG), phosphatidylcholine (PC), and phosphatidylethanolamine (PE). The fatty acid chains comprising the phospholipids may range from about 6 to about 26 carbon atoms in length, and the lipid chains may be saturated or unsaturated. Suitable fatty acid chains include (common name presented in parentheses) n-dodecanoate (laurate), n-tretradecanoate (myristate), n-hexadecanoate (palmitate), n-octadecanoate (stearate), n-eicosanoate (arachidate), n-docosanoate (behenate), n-tetracosanoate (lignocerate), cis-9-hexadecenoate (palmitoleate), cis-9-octadecanoate (oleate), cis,cis-9,12-octadecandienoate (linoleate), all cis-9, 12, 15-octadecatrienoate (linolenate), and all cis-5,8,11,14-eicosatetraenoate (arachidonate). The two fatty acid chains of a phospholipid may be identical or different. Acceptable phospholipids include dioleoyl PS, dioleoyl PC, distearoyl PS, distearoyl PC, dimyristoyl PS, dimyristoyl PC, dipalmitoyl PG, stearoyl, oleoyl PS, palmitoyl, linolenyl PS, and the like.

The phospholipids may come from any natural source, and, as such, may comprise a mixture of phospholipids. For example, egg yolk is rich in PC, PG, and PE, soy beans contains PC, PE, PI, and PA, and animal brain or spinal cord is enriched in PS. Phospholipids may come from synthetic sources too. Mixtures of phospholipids having a varied ratio of individual phospholipids may be used. Mixtures of different phospholipids may result in liposome compositions having advantageous activity or stability of activity properties. The above mentioned phospholipids may be mixed, in optimal ratios with cationic lipids, such as N-(1-(2,3-dioleolyoxy)propyl)-N,N,N-trimethyl ammonium chloride, 1,1′-dioctadecyl-3,3,3′,3′-tetramethylindocarbocyanine perchloarate, 3,3′-deheptyloxacarbocyanine iodide, 1,1′-dedodecyl-3,3,3′,3′-tetramethylindocarbocyanine perchloarate, 1,1′-dioleyl-3,3,3′,3′-tetramethylindo carbocyanine methanesulfonate, N-4-(delinoleylaminostyryl)-N-methylpyridinium iodide, or 1,1,-dilinoleyl-3,3,3′,3′-tetramethylindocarbocyanine perchloarate.

Liposomes may optionally comprise sphingolipids, in which spingosine is the structural counterpart of glycerol and one of the one fatty acids of a phosphoglyceride, or cholesterol, a major component of animal cell membranes. Liposomes may optionally, contain pegylated lipids, which are lipids covalently linked to polymers of polyethylene glycol (PEG). PEGs may range in size from about 500 to about 10,000 daltons.

Liposomes may further comprise a suitable solvent. The solvent may be an organic solvent or an inorganic solvent. Suitable solvents include, but are not limited to, dimethylsulfoxide (DMSO), methylpyrrolidone, N-methylpyrrolidone, acetronitrile, alcohols, dimethylformamide, tetrahydrofuran, or combinations thereof.

Liposomes carrying therapeutic fusion peptides of the present disclosure may be prepared by any known method of preparing liposomes for drug delivery, such as, for example, detailed in U.S. Pat. Nos. 4,241,046, 4,394,448, 4,529,561, 4,755,388, 4,828,837, 4,925,661, 4,954,345,4,957,735, 5,043,164, 5,064,655, 5,077,211 and 5,264,618, the disclosures of which are hereby incorporated by reference in their entirety. For example, liposomes may be prepared by sonicating lipids in an aqueous solution, solvent injection, lipid hydration, reverse evaporation, or freeze drying by repeated freezing and thawing. In a preferred embodiment the liposomes are formed by sonication. The liposomes may be multilamellar, which have many layers like an onion, or unilamellar. The liposomes may be large or small. Continued high-shear sonication tends to form smaller unilamellar lipsomes.

As would be apparent to one of ordinary skill, all of the parameters that govern liposome formation may be varied. These parameters include, but are not limited to, temperature, pH, concentration of methionine compound, concentration and composition of lipid, concentration of multivalent cations, rate of mixing, presence of and concentration of solvent.

In another embodiment, the therapeutic fusion peptides may be delivered to a cell as a microemulsion. Microemulsions are generally clear, thermodynamically stable solutions comprising an aqueous solution, a surfactant, and “oil.” The “oil” in this case, is the supercritical fluid phase. The surfactant rests at the oil-water interface. Any of a variety of surfactants are suitable for use in microemulsion formulations including those described herein or otherwise known in the art. The aqueous microdomains suitable for use in the invention generally will have characteristic structural dimensions from about 5 nm to about 100 nm. Aggregates of this size are poor scatterers of visible light and hence, these solutions are optically clear. As will be appreciated by a skilled artisan, microemulsions can and will have a multitude of different microscopic structures including sphere, rod, or disc shaped aggregates. In one embodiment, the structure may be micelles, which are the simplest microemulsion structures that are generally spherical or cylindrical objects. Micelles are like drops of oil in water, and reverse micelles are like drops of water in oil. In an alternative embodiment, the microemulsion structure is the lamellae. It comprises consecutive layers of water and oil separated by layers of surfactant. The “oil” of microemulsions optimally comprises phospholipids. Any of the phospholipids detailed above for liposomes are suitable for embodiments directed to microemulsions. The composition of the invention may be encapsulated in a microemulsion by any method generally known in the art.

In yet another embodiment, the therapeutic fusion peptide may be delivered in a dendritic macromolecule, or a dendrimer. Generally speaking, a dendrimer is a branched tree-like molecule, in which each branch is an interlinked chain of molecules that divides into two new branches (molecules) after a certain length. This branching continues until the branches (molecules) become so densely packed that the canopy forms a globe. Generally, the properties of dendrimers are determined by the functional groups at their surface. For example, hydrophilic end groups, such as carboxyl groups, would typically make a water-soluble dendrimer. Alternatively, phospholipids may be incorporated in the surface of a dendrimer to facilitate absorption across the skin. Any of the phospholipids detailed for use in liposome embodiments are suitable for use in dendrimer embodiments. Any method generally known in the art may be utilized to make dendrimers and to encapsulate compositions of the invention therein. For example, dendrimers may be produced by an iterative sequence of reaction steps, in which each additional iteration leads to a higher order dendrimer. Consequently, they have a regular, highly branched 3D structure, with nearly uniform size and shape. Furthermore, the final size of a dendrimer is typically controlled by the number of iterative steps used during synthesis. A variety of dendrimer sizes are suitable for use in the invention. Generally, the size of dendrimers may range from about 1 nm to about 100 nm.

### Administration

In certain aspects, a therapeutically effective amount of the therapeutic fusion peptides of the present disclosure may be administered to a subject. Administration is performed using standard effective techniques, including peripherally (i.e. not by administration into the central nervous system) or locally to the central nervous system. Peripheral administration includes but is not limited to intravenous, intraperitoneal, subcutaneous, pulmonary, transdermal, intramuscular, intranasal, buccal, sublingual, or suppository administration. Local administration, including directly into the central nervous system (CNS) includes but is not limited to via a lumbar, intraventricular or intraparenchymal catheter or using a surgically implanted controlled release formulation. Pheresis may be used to deliver the therapeutic fusion peptides of the present disclosure. In certain embodiments, the therapeutic fusion peptides of the present disclosure may be administered via an infusion (continuous or bolus).

Pharmaceutical compositions for effective administration are deliberately designed to be appropriate for the selected mode of administration, and pharmaceutically acceptable excipients such as compatible dispersing agents, buffers, surfactants, preservatives, solubilizing agents, isotonicity agents, stabilizing agents and the like are used as appropriate. Remington's Pharmaceutical Sciences, Mack Publishing Co., Easton Pa., 16Ed ISBN: 0-912734 Apr. 3, latest edition, incorporated herein by reference in its entirety, provides a compendium of formulation techniques as are generally known to practitioners.

Effective peripheral systemic delivery by intravenous or intraperitoneal or subcutaneous injection is a preferred method of administration to a living patient. Suitable vehicles for such injections are straightforward. In addition, however, administration may also be effected through the mucosal membranes by means of nasal aerosols or suppositories. Suitable formulations for such modes of administration are well known and typically include surfactants that facilitate cross-membrane transfer. Such surfactants are often derived from steroids or are cationic lipids, such as N-[1-(2,3-dioleoyl)propyl]-N,N,N-trimethyl ammonium chloride (DOTMA) or various compounds such as cholesterol hemisuccinate, phosphatidyl glycerols and the like.

For therapeutic applications, a therapeutically effective amount of the therapeutic fusion peptides of the present disclosure is administered to a subject. A “therapeutically effective amount” is an amount of the therapeutic composition sufficient to produce a measurable response (e.g., tumor regression). Actual dosage levels of active ingredients in a therapeutic composition of the invention can be varied so as to administer an amount of the active compound(s) that is effective to achieve the desired therapeutic response for a particular subject. The selected dosage level will depend upon a variety of factors including the activity of the therapeutic composition, formulation, the route of administration, combination with other drugs or treatments, tumor size and longevity, infection, and the physical condition and prior medical history of the subject being treated. In some embodiments, a minimal dose is administered, and dose is escalated in the absence of dose-limiting toxicity. Determination and adjustment of a therapeutically effective dose, as well as evaluation of when and how to make such adjustments, are known to those of ordinary skill in the art of medicine.

The frequency of dosing may be once, twice, three times or more daily or once, twice, three times or more per week or per month, as needed as to effectively treat the symptoms or disease. In certain embodiments, the frequency of dosing may be once, twice or three times daily. For example, a dose may be administered every 24 hours, every 12 hours, or every 8 hours. In a specific embodiment, the frequency of dosing may be twice daily.

In any of the foregoing embodiments, the pharmaceutical composition can be administered at a dose between about 0.1 μg/Kg and about 50 μg/Kg of the polypeptide of the present disclosure per patient body weight. By way of further example, but not limitation, the pharmaceutical composition can be administered at a dose between about 0.1 μg/Kg and about 50 μg/Kg, about 0.1 μg/Kg to about 25 μg/Kg, about 0.1 μg/Kg to about 10 μg/Kg, about 1 μg/Kg to about 50 μg/Kg, about 1 μg/Kg to about 25 μg/Kg, about 1 μg/Kg to about 10 μg/Kg, about 10 μg/Kg to about 50 μg/Kg, about 25 μg/Kg to about μg/Kg, about 0.1 μg/Kg, about 0.5 μg/Kg, about 1 μg/Kg, about 5 μg/Kg, about 10 μg/Kg, about 15 μg/Kg, about 20 μg/Kg, about 25 μg/Kg, about 30 μg/Kg, about 35 μg/Kg, about 40 μg/Kg, about 45 μg/Kg, or about 50 μg/Kg of the polypeptide of the present disclosure per patient body weight.

Duration of treatment could range from a single dose administered on a one-time basis to a life-long course of therapeutic treatments. The duration of treatment can and will vary depending on the subject and the cancer to be treated. For example, the duration of treatment may be for 1 day, 2 days, 3 days, 4 days, 5 days, 6 days, or 7 days. Or, the duration of treatment may be for 1 week, 2 weeks, 3 weeks, 4 weeks, 5 weeks or 6 weeks. Alternatively, the duration of treatment may be for 1 month, 2 months, 3 months, 4 months, 5 months, 6 months, 7 months, 8 months, 9 months, 10 months, 11 months, or 12 months. In still another embodiment, the duration of treatment may be for 1 year, 2 years, 3 years, 4 years, 5 years, or greater than 5 years. It is also contemplated that administration may be frequent for a period of time and then administration may be spaced out for a period of time. For example, duration of treatment may be 5 days, then no treatment for 9 days, then treatment for 5 days.

The timing of administration of the treatment relative to the disease itself and duration of treatment will be determined by the circumstances surrounding the case. Treatment could begin immediately, such as at the time of diagnosis, or treatment could begin following surgery. Treatment could begin in a hospital or clinic itself, or at a later time after discharge from the hospital or after being seen in an outpatient clinic.

### EXAMPLES

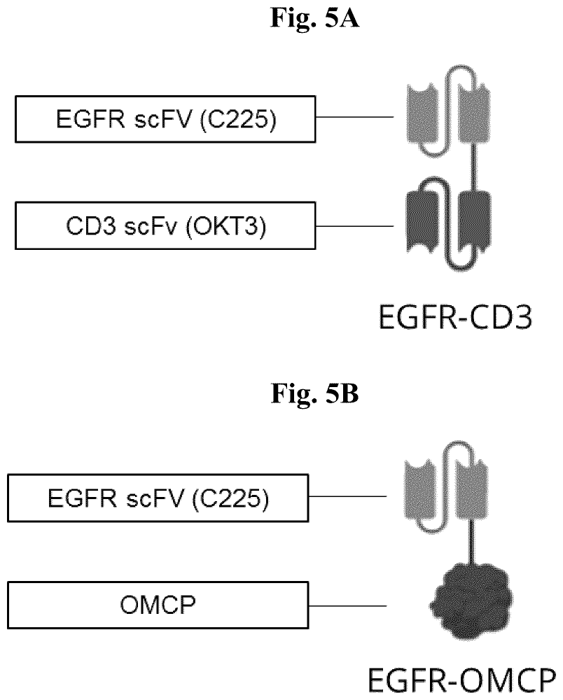

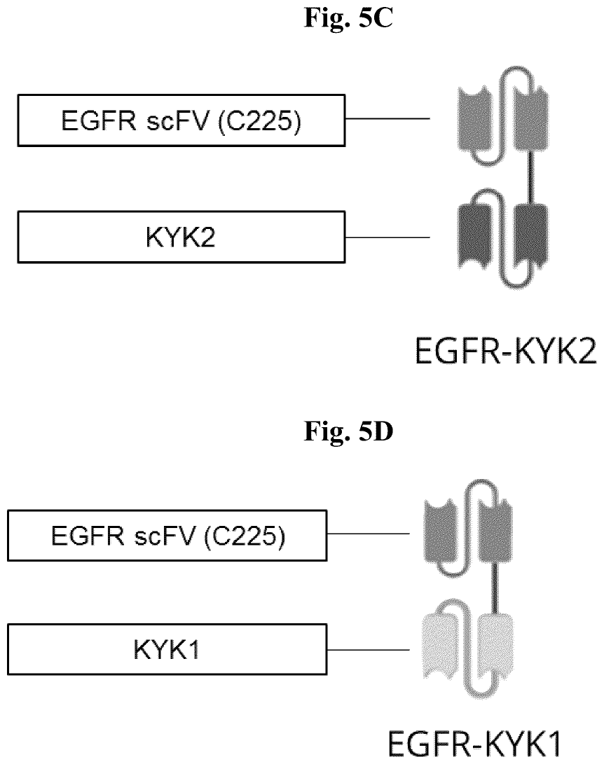

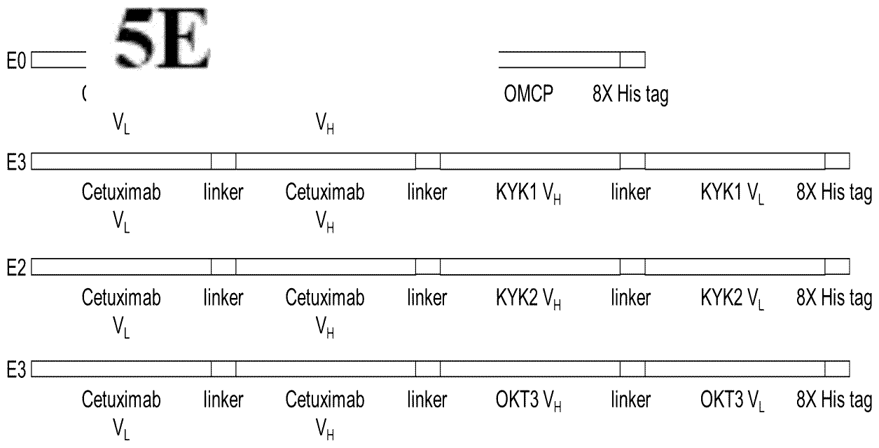

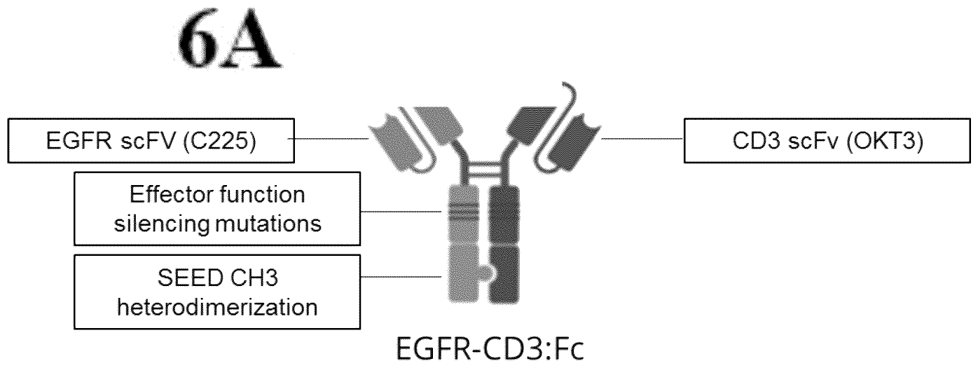

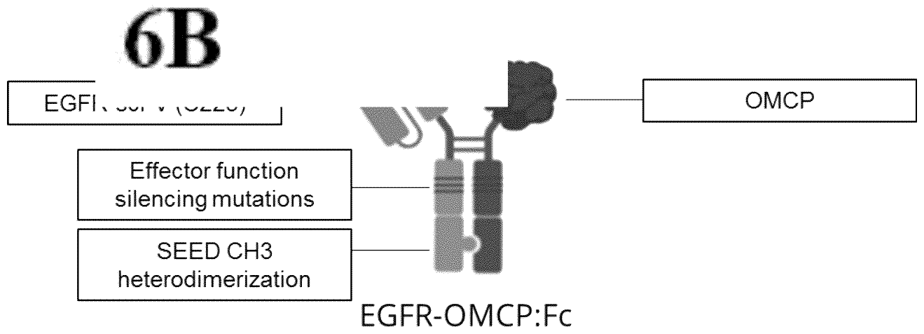

In the following examples, the designations E0, E1, E2 and E3 refer to the constructs in the table below which are also schematically depicted in FIGS. 5A-5E and have the sequences as disclosed below. These constructs were designed in silico using standard approaches well-known in the art. Finalized sequences were codon optimization prior to DNA synthesis followed by expression in CHO cells and purification from culture media by nickel chromatography and PBS buffer exchange at pH 7.

| Designation | Description | Seq. ID No. | FIG. |
|---|---|---|---|
| E0 | EGFR-OMCP or | 18 | 5B, 5E |
| | OMCP-EGFR | | |
| E1 | EGFR-KYK1 or | 20 | 5D, 5E |
| | KYK1-EGFR | | |
| E2 | EGFR-KYK2 or | 19 | 5C, 5E |
| | KYK2-EGFR | | |
| E3 | EGFR-CD3 or | 16 | 5A, 5E |
| | CD3-CGFR or | | |
| | EGFR-OKT3 or | | |
| | OKT3-EGFR | | |

### Example 1—Binding Affinity of Anti-EGFR Bispecific Fusion Proteins

Four constructs with distinct immune-targeting moieties were designed: (E0) OMCP, a viral NKG2D ligand, (E1) single-chain variable fragment (scFv) of the anti-NKG2D antibody KYK1, (E2) scFv of the anti-NKG2D antibody KYK2, or as a positive control, (E3) scFv of the anti-CD3 antibody OKT3. Each immune-targeting domain was linked via a glycine-serine linker to a scFv of the anti-EGFR antibody Cetuximab. Cetuximab was chosen for these proof-of-concept studies because it is off-patent and has established bispecific therapeutic functionality in a range of tumor models.

FIGS. 5A-5D depict scFv constructs which were designed and tested. Specifically, FIG. 5A depicts a bi-specific fusion protein comprising the amino acid sequence set forth in SEQ ID NO: 16 (DILLTQSPVILSVSPGERVSFSCRASQSIGTNIHWYQQRTNGSPRLLIKYASESISGIPSRFS GSGSGTDFTLSINSVESEDIADYYCQQNNNWPTTFGAGTKLELKRTVAGGGGSGGGGSG GGGSQVQLKQSGPGLVQPSQSLSITCTVSGFSLTNYGVHWVRQSPGKGLEWLGVIWSG GNTDYNTPFTSRLSINKDNSKSQVFFKMNSLQSNDTAIYYCARALTYYDYEFAYWGQG TLVTVSAGGGGSDIKLQQSGAELARPGASVKMSCKTSGYTFTRYTMHWVKQRPGQGL EWIGYINPSRGYTNYNQKFKDKATLTTDKSSSTAYMQLSSLTSEDSAVYYCARYYDDH YCLDYWGQGTTLTVSSVEGGSGGSGGSGGSGGVDDIQLTQSPAIMSASPGEKVTMTCR ASSSVSYMNWYQQKSGTSPKRWIYDTSKVASGVPYRFSGSGSGTSYSLTISSMEAEDAA TYYCQQWSSNPLTFGAGTKLELKHHHHHHHH). The fusion protein in FIG. 5A comprises an scFv of an antibody to EGFR (cetuximab—SEQ ID NO: 14) coupled to CD3 scFv where the CD3 scFv comprises the amino acid sequence of SEQ ID NO: 17 (DIKLQQSGAELARPGASVKMSCKTSGYTFTRYTMHWVKQRPGQGLEWIGYINPSRGYT NYNQKFKDKATLTTDKSSSTAYMQLSSLTSEDSAVYYCARYYDDHYCLDYWGQGTTL TVSSVEGGSGGSGGSGGSGGVDDIQLTQSPAIMSASPGEKVTMTCRASSSVSYMNWYQ QKSGTSPKRWIYDTSKVASGVPYRFSGSGSGTSYSLTISSMEAEDAATYYCQQWSSNPL TFGAGTKLELK) via a linker comprising GGGGS (SEQ ID NO: 37) and including a histidine tag (HHHHHHHH (SEQ ID NO: 38)) on the c-terminal end.

FIG. 5B depicts a bi-specific fusion protein comprising the amino acid sequence set forth in SEQ ID NO: 18 (DILLTQSPVILSVSPGERVSFSCRASQSIGTNIHWYQQRTNGSPRLLIKYASESISGIPSRFS GSGSGTDFTLSINSVESEDIADYYCQQNNNWPTTFGAGTKLELKRTVAGGGGSGGGGSG GGGSQVQLKQSGPGLVQPSQSLSITCTVSGFSLTNYGVHWVRQSPGKGLEWLGVIWSG GNTDYNTPFTSRLSINKDNSKSQVFFKMNSLQSNDTAIYYCARALTYYDYEFAYWGQG TLVTVSAGGGGSGGGGSGGGGSHKLAFNFNLEINGSDTHSTVDVYLDDSQIITFDGKDIR PTIPFMIGDEIFLPFYKNVFSEFFSLFRRVPTSTPYEDLTYFYECDYTDNKSTFDQFYLYNG EEYTVKTQEATNKNMWLTTSEFRLKKWFDGEDCIMHLRSLVRKMEDSKRNTGHHHHH HHH). The fusion protein in FIG. 5B comprises an scFv of an antibody to EGFR (cetuximab—SEQ ID NO: 14) coupled to OMCP (SEQ ID NO: 1) via a linker comprising three repeats of the amino acid sequence GGGGS (SEQ ID NO: 37) and including a histidine tag (HHHHHHHH (SEQ ID NO: 38)) on the c-terminal end.

FIG. 5C depicts a bi-specific fusion protein comprising the amino acid sequence set forth in SEQ ID NO: 19 (DILLTQSPVILSVSPGERVSFSCRASQSIGTNIHWYQQRINGSPRLLIKYASESISGIPSRFS GSGSGTDFTLSINSVESEDIADYYCQQNNNWPTTFGAGTKLELKRTVAGGGGSGGGGSG GGGSQVQLKQSGPGLVQPSQSLSITCTVSGFSLTNYGVHWVRQSPGKGLEWLGVIWSG GNTDYNTPFTSRLSINKDNSKSQVFFKMNSLQSNDTAIYYCARALTYYDYEFAYWGQG TLVTVSAGGGGSQVQLVESGGGLVKPGGSLRLSCAASGFTFSSYGMHWVRQAPGKGLE WVAFIRYDGSNKYYADSVKGRFTISRDNSKNTLYLQMNSLRAEDTAVYYCAKDRGLG DGTYFDYWGQGTTVTVSSGGGGSGGGGSGGGGSQSALTQPASVSGSPGQSITISCSGSSS NIGNNAVNWYQQLPGKAPKLLIYYDDLLPSGVSDRFSGSKSGTSAFLAISGLQSEDEAD YYCAAWDDSLNGPVFGGGTKLTVLHHHHHHHH). The fusion protein in FIG. 5C comprises an scFv of an antibody to EGFR (cetuximab-SEQ ID NO: 14) coupled to KYK-2 (SEQ ID NO: 11) via a linker comprising the amino acid sequence GGGGS (SEQ ID NO: 37) and including a histidine tag (HHHHHHHH (SEQ ID NO: 38)) on the c-terminal end.

FIG. 5D depicts a bi-specific fusion protein comprising the amino acid sequence set forth in SEQ ID NO: 20 (DILLTQSPVILSVSPGERVSFSCRASQSIGTNIHWYQQRINGSPRLLIKYASESISGIPSRFS GSGSGTDFTLSINSVESEDIADYYCQQNNNWPTTFGAGTKLELKRTVAGGGGSGGGGSG GGGSQVQLKQSGPGLVQPSQSLSITCTVSGFSLTNYGVHWVRQSPGKGLEWLGVIWSG GNTDYNTPFTSRLSINKDNSKSQVFFKMNSLQSNDTAIYYCARALTYYDYEFAYWGQG TLVTVSAGGGGSEVQLVESGGGVVQPGGSLRLSCAASGFTFSSYGMHWVRQAPGKGLE WVAFIRYDGSNKYYADSVKGRFTISRDNSKNTLYLQMNSLRAEDTAVYYCAKDRFGY YLDYWGQGTLVTVSSGGGGSGGGGSGGGGSQPVLTQPSSVSVAPGETARIPCGGDDIET KSVHWYQQKPGQAPVLVIYDDDDRPSGIPERFFGSNSGNTATLSISRVEAGDEADYYCQ VWDDNNDEWVFGGGTQLTVLHHHHHHHH). The fusion protein in FIG. 5D comprises an scFv of an antibody to EGFR (cetuximab—SEQ ID NO: 14) coupled to KYK-1 (SEQ ID NO: 9) via a linker comprising the amino acid sequence GGGGS (SEQ ID NO: 37) and including a histidine tag (HHHHHHHH (SEQ ID NO: 38)) on the c-terminal end.

To confirm that all bispecific antibodies interact with their target receptors, their interactions were measured using surface plasmon resonance. A ProteOn XPR36 instrument (Bio-Rad) was used to determine the kinetics of protein: protein interactions. All experiments were carried out at a flow rate of 100 μl/min, 25° C., and in running buffer containing 1X PBS, pH 7.4, 0.005% Tween 20. GLC chips were activated with 1-Ethyl-3-(3-dimethylaminopropyl) carbodiimide (EDC)/N-Hydroxysuccinimide (NHS) for amine coupling of proteins. On one chip ˜1000 RUs of human NKG2D were coupled. ˜500 RUs of each bispecific antibody was coupled to a second chip. Ethanolamine was then used to quench unreacted esters.

Bispecific antibody binding to human NKG2D was determined over a range of 300-0.38 nM. Human NKG2D binding was regenerated with pulses of 10 mM HCl. EGF-FcR binding to bispecific antibodies was determined over a range of 9-0.1 nM. Data was analyzed using ProteOn analysis software with bispecific antibody: NKG2D curves fitted using a 1:1 langmuir binding model and EGFR-Fc:bispecific antibody curves fitted using a bivalent binding model.

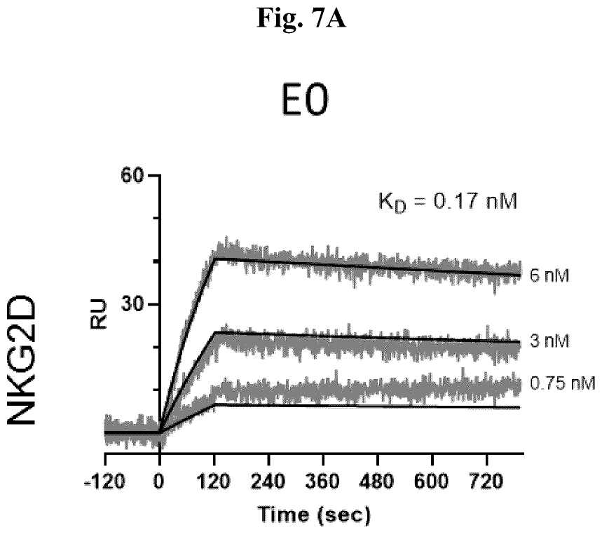

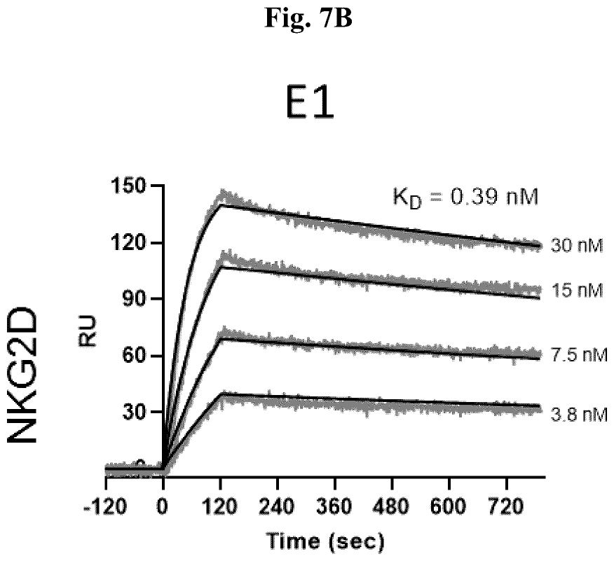

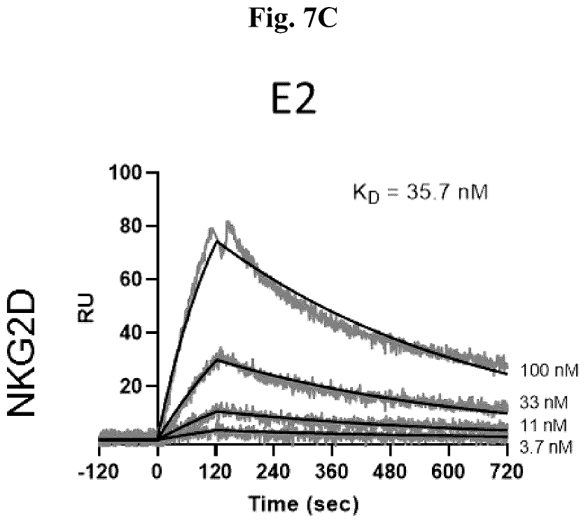

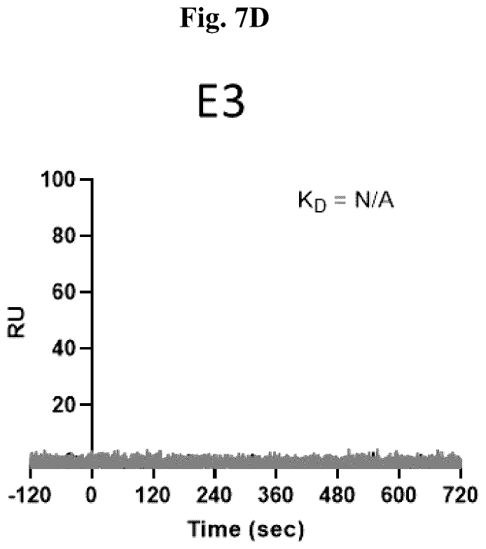

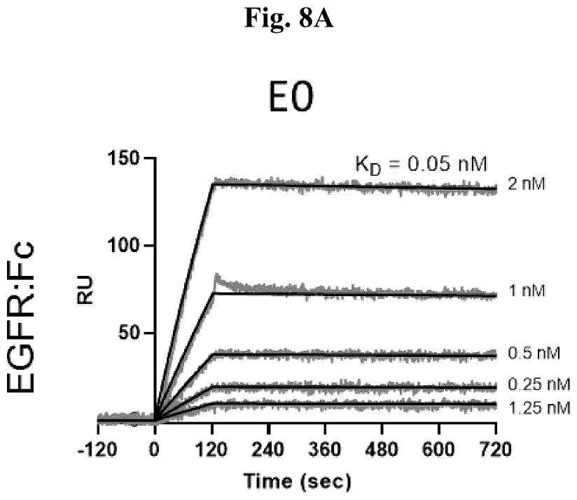

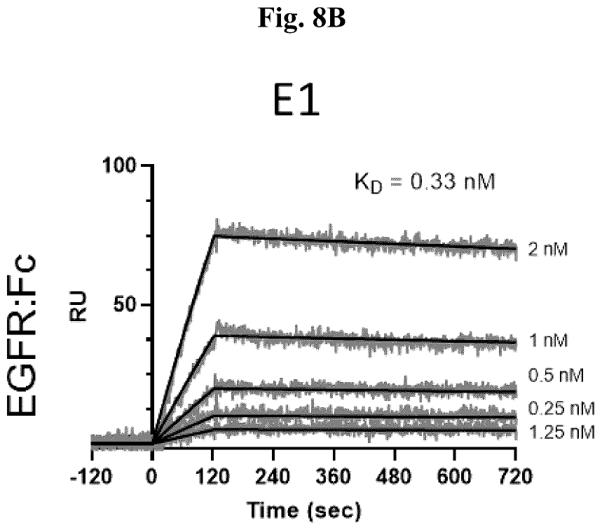

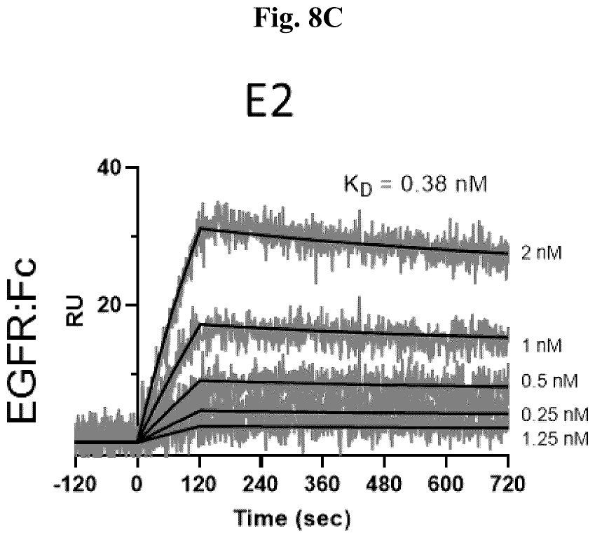

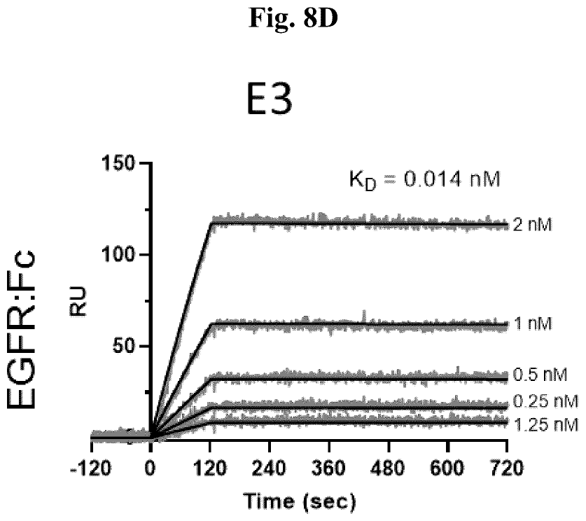

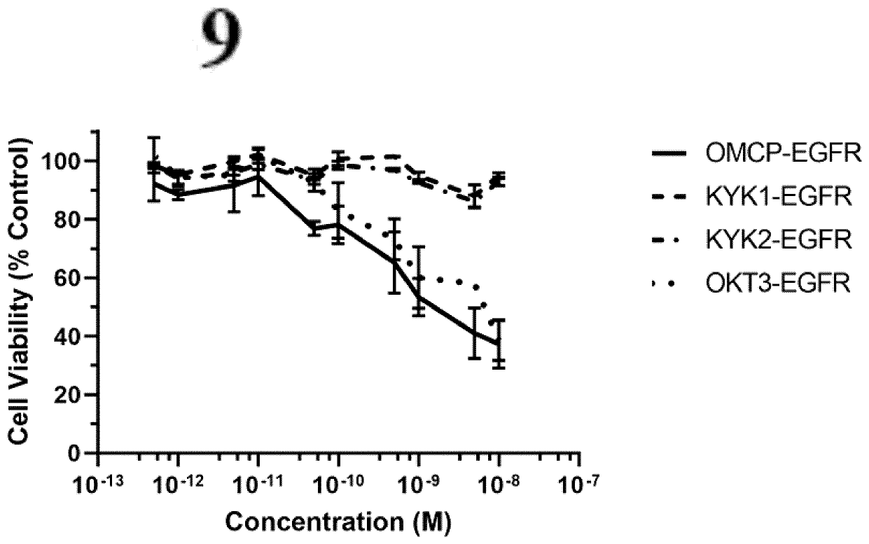

The resulting plasmon resonance measurements for NKG2D binding are shown in FIGS. 7A-7D for E0, E1, E2 and E3, respectively while the resulting plasmon resitance measurements for EGFR-Fc binding are shown in FIGS. 8A-8D for E0, E1, E2 and E3, respectively. Processed data points are shown in gray while the fitted model is shown in black in FIGS. 7A-7D and FIGS. 8A-8D.

As shown in FIGS. 7A-7D, all bispecific fusion proteins with a NKG2D-targeting domain (E0, E1 and E2) bound with high affinity to NKG2D. As expected, E3 did not bind to NKG2D since the OKT3 scFv is specific for CD3. E0 bound to NKG2D with a KD of 0.17 nM similarly to the affinity of OMCP for NKG2D (0.2 nM) (Lazear et al., Crystal Structure of the Cowpox Virus-Encoded NKG2D Ligand OMCP, J. Virol 87 (2): 840-850 (2013)). E2 bound to NKG2D with a KD of 35.7 nM, similarly to the affinity of the KYK1 antibody to NKG2D (27 nM) (Kwong, Generation, affinity maturation, and characterization of a human anti-human NKG2D monoclonal antibody with dual antagonistic and agonistic activity, J. Mol. Bio. 384 (5): 1143-1156 (2008)). E1 bound with a higher affinity then previously reported (KD 0.39 nM vs 5.8). No mass transport or other confounding variables were detected. While the affinity is higher than reported, the higher affinity of E1 does not limit its use in these experiments.

As shown in FIGS. 8A-8D, all bispecific fusion proteins containing an EGFR-targeting domain bound with high affinity to EGFR-Fc.

All bispecific antibodies contain an identical tumor-targeting scFv from Cetuximab which is specific for EGFR. All bispecific antibodies bound to EGFR-Fc. E1 and E2 bound with KD of 0.33 and 0.38 nM in close agreement with the published affinity of Cetuximab (0.4 nM). E0 and E3 bound EGFR-Fc with higher apparent affinities then expected, though both sensograms show evidence of mass transport which limits the confidence in these measured affinities. In the absence of mass transport limitations, the affinity of E0 and E3 would be expected to be similar to Cetuximab, E1, and E2. Regardless, in all cases the bispecific antibodies show high affinity binding to their intended receptors. Therefore confirming that the bispecific antibodies have the intended receptor-targeting.

### Example 2—In Vitro Cytotoxicity of Anti-EGFR Bispecific Fusion Proteins

Human PBMCs from non-smoker donors (AllCells, frozen vials) were plated in 96 well plates at a 10:1 ratio with MDA-MB-231 breast cancer cells in the presence of limiting dilutions of test agents or negative controls. The assay media was RPMI media with 50 μM betα-mercapatoethanol and 5% heat inactivated FBS. MDA-MB-231 cells were grown in standard media (high-glucose DMEM, 10% heat inactivated FBS) to 70-80% confluency prior to assay initiation and were collected using Accutase to preserve surface protein expression. Cells were incubated for 48 hours, then imaged. All media was removed and wells were washed 3X with 200 μL PBS to remove immune cells prior to incubation with 100 μL PBS and 100 μL CellTiterGlo2.0 (Promega) with shaking in the dark for 10 minutes and luciferase output measured per the manufacturer's instructions, which directly correlates to viable cell numbers. All calculations are reported in comparison to test-agent negative controls.

FIG. 9 shows the cell viability for each treatment as a function of concentration.

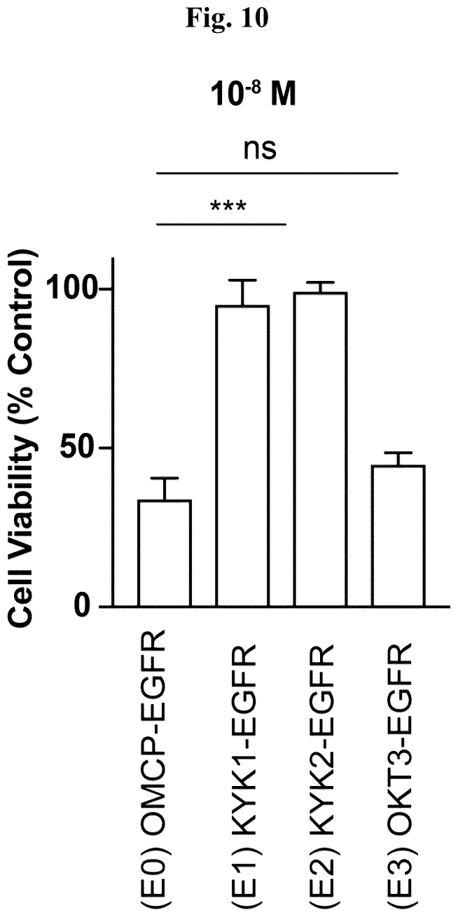

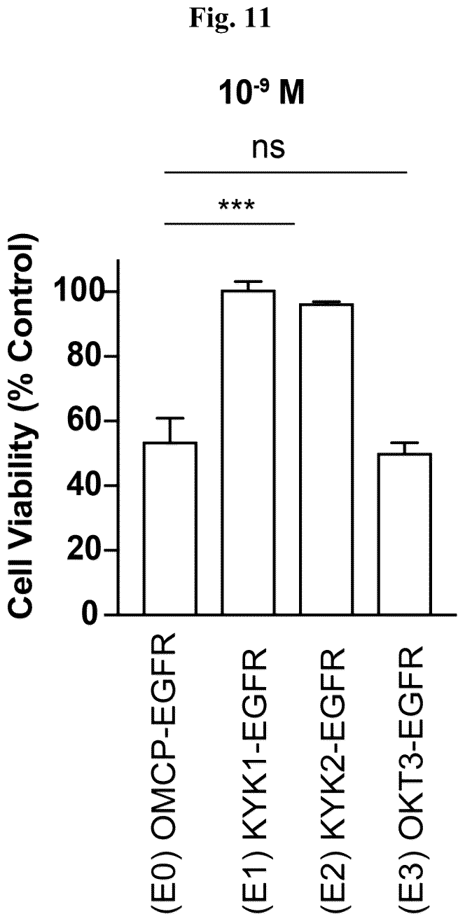

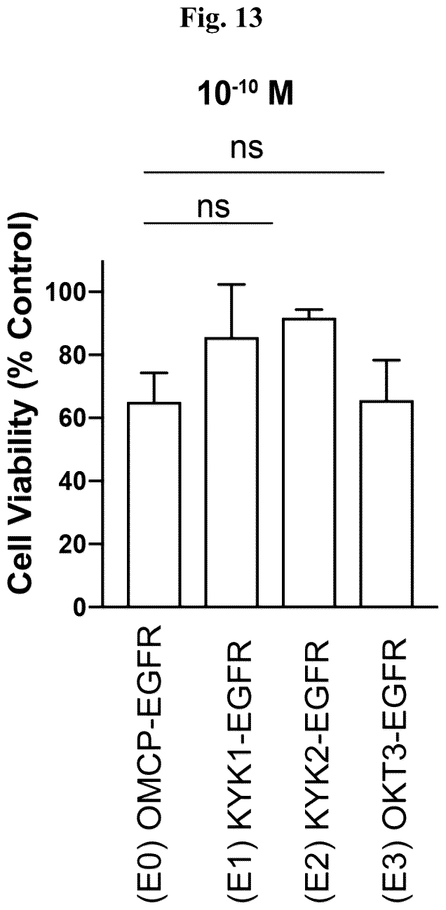

FIGS. 10, 11 and 13 show the cell viability for the 1×10⁻⁸ M, 1×10⁻⁹ M and 1×10⁻¹⁰ M treatments for each group, respectively.

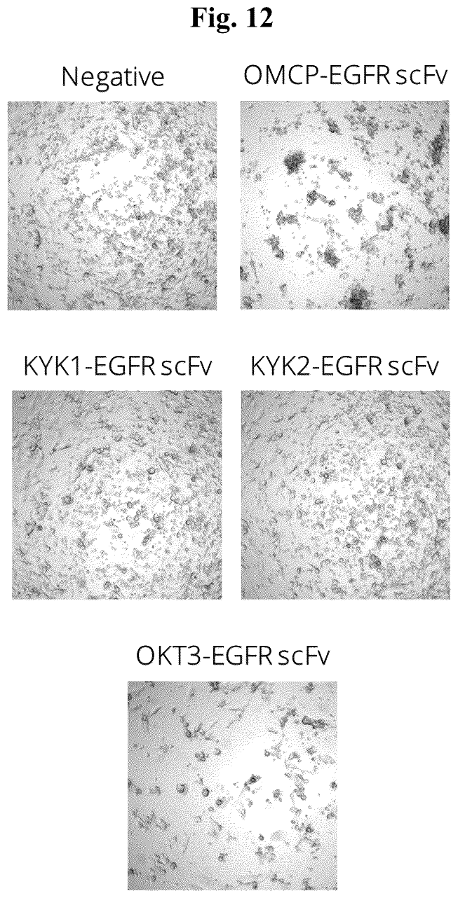

FIG. 12 shows images of the cells for the negative control (no construct added) and for treatment groups receiving 1 nM of the bispecific fusion proteins.

As demonstrated by FIGS. 9-11 and 13, both E0 (OMCP-EGFR) and E3 (OKT3-EGFR) markedly decrease cell viability in the presence of PBMCs, suggesting that OMCP is similarly or slightly more efficient than anti-CD3 at inducing cytotoxicity. Interestingly, the anti-NKG2D antibodies KYK1 and KYK2 do not significantly affect cell viability in the presence of PBMCs.

As demonstrated by FIG. 12, the negative control shows broad growth of MDA-MB-231 cells with PBMC cells overlaid. The anti-NKG2D bispecifics (KYK1-EGFR scFv (E1) and KYK2-EGFR scFv (E2)) seem to have little effect, but OMCP-EGFR scFv (E0) (which also binds NKG2D) results in dramatic clearance of MDA-MB-231 cells and generation of immune-cell activation clusters. The anti-CD3 bispecific (OKT3-EGFR scFv (E3)) also resulted in clearance of MDA-MB-231 cells, but with reduced presence of immune-activation clusters.

As expected, the anti-CD3 containing construct OKT3-EGFR induced MDA-MB-231 cell death inversely correlated to the construct concentration. However, a disparity was noted between the NKG2D binding constructs. While the KYK1 and KYK2 anti-NKG2D antibody containing constructs did not induce significant cell death, the OMCP-EGFR construct induced cell death similarly or slightly better than the control OKT3-EGFR constructs. Visualization of wells showed significant immune cell cluster formation, indicative of cell activation, in the OMCP-EGFR wells but not the KYK1-EGFR or KYK2-EGFR wells, supporting the cell viability assay results. These data suggest that OMCP-EGFR uniquely enhances PBMC-target cell death despite binding the same NKG2D receptor.

### Example 3—In Vitro Cytotoxicity of Anti-EGFR Bispecific Fusion Proteins

Fresh human nonsmoker PBMCs were plated in 96 well plates at a 5:1 ratio with A549 lung cancer cells in the presence of limiting dilutions of test agents (E0, E1, E2 and E3) or negative controls. Target cells were labeled with cell trace violet (CTV) dye (ThermoFischer) prior to incubation overnight with PBMCs, followed by flow cytometric analysis for cell viability. The test agents were bispecific proteins E0, E1, E2 and E3 with an anti-EGFR scFv (derived from Cituximab) and an immune-specific domain, joined by a ser-gly linker to OMCP (E0), KYK1 anti-NKG2D scFv (E1), KYK2 anti-NKG2D scFv (E2), or OKT3 anti-CD3 scFv (E3) as described above.

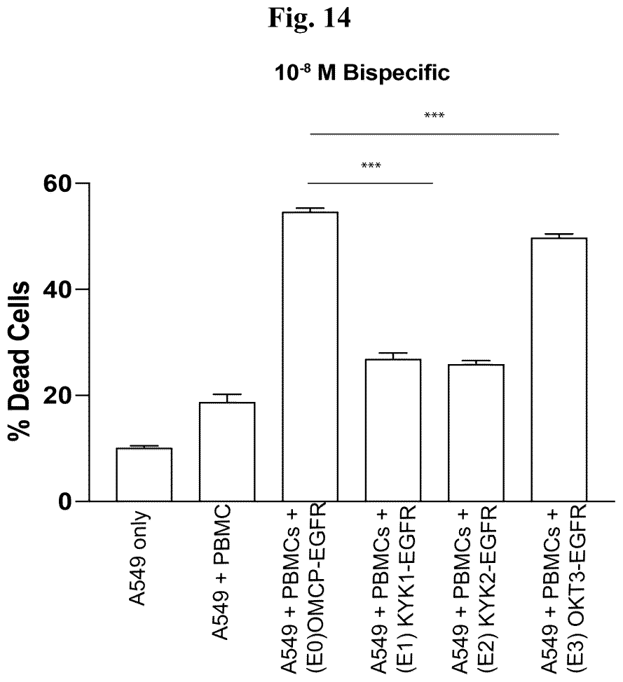

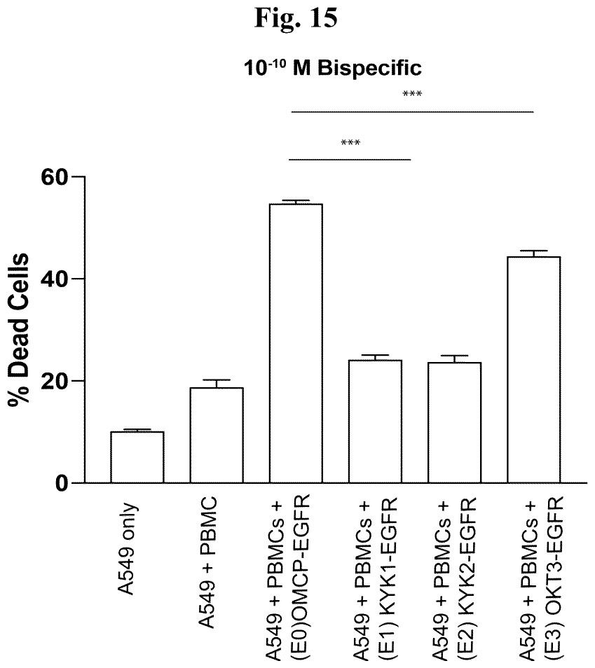

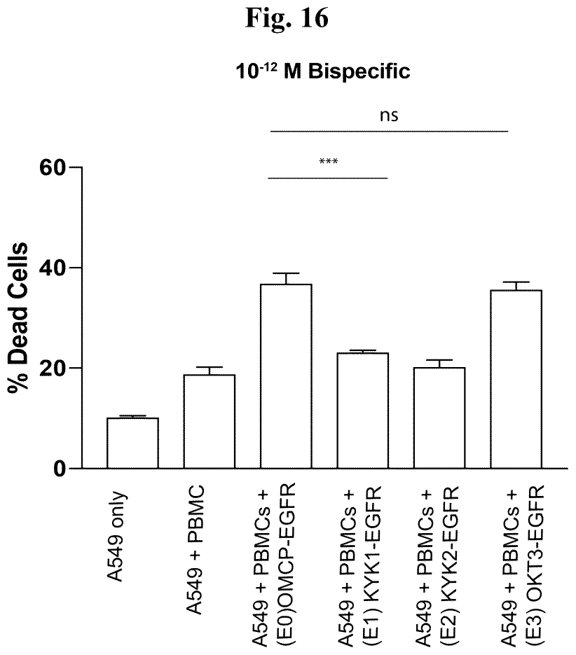

FIGS. 14-16 show the resulting cell killing data at 1×10⁻⁸ M, 1×10⁻¹⁰ M, and 1×10⁻¹² M concentrations of the test agents, respectively.

As demonstrated by FIGS. 14-16, both OMCP (which binds NKG2D) and the OKT3 (which binds CD3) bispecific test agents (E0 and E3, respectively) measurably increase A549 cell death at concentrations as low as 1 pM (10e-12 M). However, anti-NKG2D binding constructs (KYK1-EGFR (E1) and KYK2-EGFR (E2)) do not create a measurable effect over PBMC cells alone.

The data here suggest that OMCP-EGFR bispecifics enhance PBMC killing of A549 cells to a similar or greater degree than the anti-CD3 OKT3-EGFR controls. However, NKG2D binding KYK1-EGFR and KYK2-EGFR constructs do not induce significant cell killing. These data are consistent to those seen in the MDA-MB-231 cell killing assay using an alternative assay format, suggesting that the results are not isolated to a single cell line or measurement technique.

### Example 4: In Vitro Cytotoxicity of Anti-EGFR Bispecific Fusion Proteins With Isolated NK and CD8⁺ T cells

Fresh human nonsmoker PBMCs were purified into either NK or CD8+ T cell fractions using standard magnetic bead isolation kits (Miltenyibiotec Inc.). NK or CD8+ T cells were plated at a concentration designed to replicate their relative proportions of the 5:1 effector: target ratio of bulk PBMC assay described above. Thus NK cells, which make up 20% of PBMCS, were incubated at 1:1 effetcor: target ratio, while CD8+ T cells, which are 50% of PBMCs, were incubated at a 2.5:1 effector: target ratio. The target A549 cells were labeled with cell trace violet (CTV) (ThermoFischer) dye prior to incubation overnight with NK or CD8+ T cells in the presence or absence of bispecific anitbodies, followed by flow cytometric analysis for cell viability.

E0, E1, E2, and E3 were tested at a concentration of 10⁻¹⁰ M (100 pM).

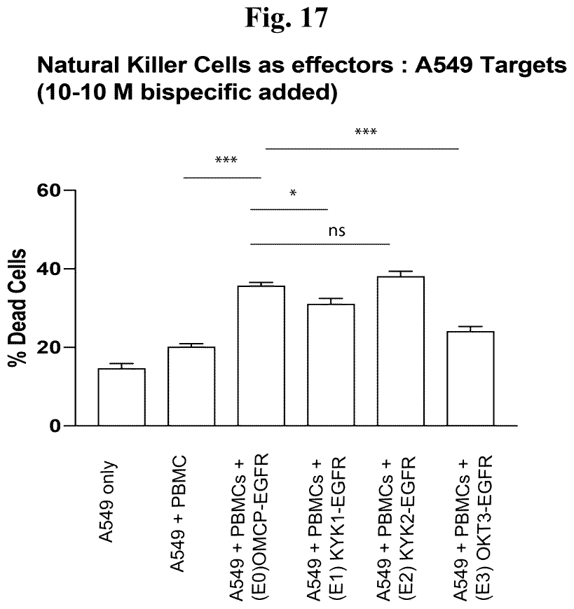

FIG. 17 shows NK cell killing for each of the constructs tested as well as A549 only, and A549 plus NK cell controls.

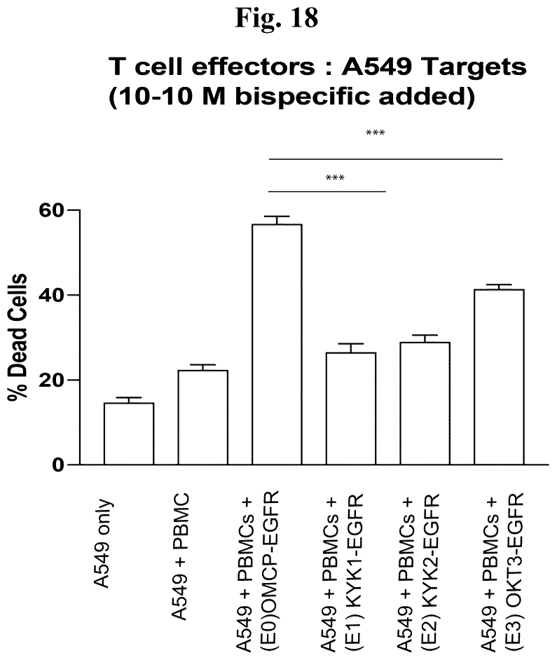

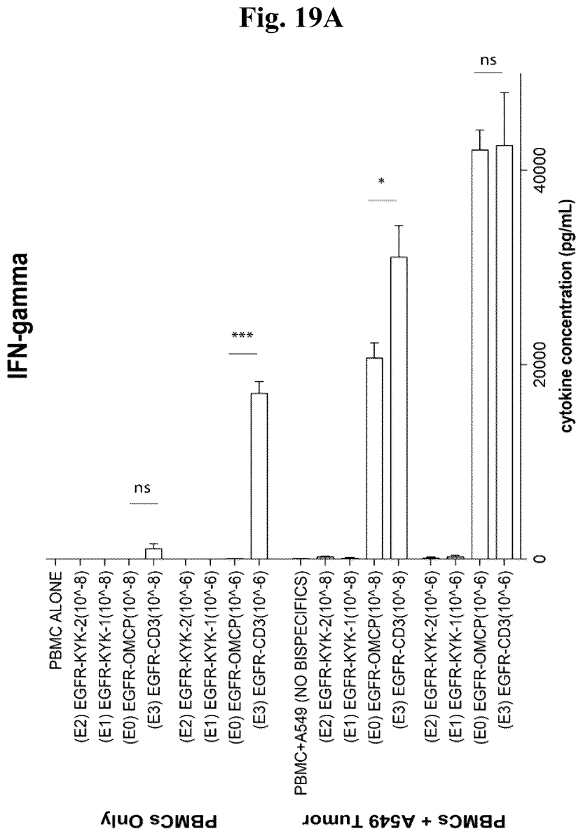

FIG. 18 shows T cell killing for each of the constructs tested as well as A549 only, and A549 plus NK cell controls.

As expected, each NKG2D-binding bispecific (OMCP-EGFR, KYK1-EGFR, and KYK2-EGFR) enhanced NK cell anti-tumor cytotoxicity due to their ability to engage the activating receptor NKG2D. As expected, the OKT3-EGFR construct, which binds the T cell receptor CD3, has no significant effect on NK cell function but does enhance T cell cytotoxicity.

The activating receptor NKG2D is also expressed on CD8⁺ T cells (as well as other cell populations such as NKT cells and gamma delta T cells) (Roulet DH, Roles of the NKG2D Immunoreceptor and its Ligands, Nature Review Immunology 3:781-790 (2003)). This broad-based expression on multiple types of cytotoxic lymphocytes makes it a unique ligand to target for broad activation of cytotoxicity across multiple cell types capable of tumor killing. Interestingly using purified CD8⁺ T cell cultures the OMCP-EGFR construct significantly enhanced CD8⁺ T cell cytotoxicity and even exceeded the functionality of the anti-CD3 OKT3-EGFR control. The use of KYK1-EGFR (E1) or KYK2-EGFR (E2) bispecific engagers did not enhance cytotoxicity over CD8⁺ T cells alone. Taken together we can conclude that enhanced function of OMCP containing constructs may be due to the: 1) high affinity binding of OMCP to NKG2D; 2) coupled with lack of receptor internalization (see Campbell JA, Zoonotic orthopoviruses encode a high-affinity antagonist of NKG2D, J. Exp. Med. 204 (6): 1311-1317 (2007)). However our data points out that: 1) the use of anti-NK2D targeting allows for improved killing of targets over CD3 targeting due to engagement of not just T cells but NK cells as well; 2) the use of OMCP improves NKG2D bi-specific targeting over the use of established KYK1 and KYK2 antibodies.

### Example 5: In Vitro Cytokine Release Analysis

The use of anti-CD3 bispecifics has been complicated by side effects of cytokine release syndrome (https://www.ncbi.nlm.nih.gov/pmc/articles/PMC6003181/#). Since the engagement and crosslinking of the T cells receptor in the form of CD3 antibody can activate all T cells systemically (CD4⁺ or CD8⁺), not just at the site of the tumor, cytokine release syndrome, or cytokine storm has resulted in unexpected morbidity and mortality. In theory, NKG2D engagement should result in lower levels of such complications since NKG2D acts as a co-stimulatory rather than a primary-stimulating activating receptor on T cells. In addition NKG2D expression is most prominent on memory and effector CD8⁺ T cells. Thus its engagement should not result in broad stimulation of naïve and antigen inexperienced T cells (https://doi.org/10.1371/journal.pone.0012635) or of CD4 ⁺ T cells. The use of monomeric OMCP offers one more advantage over the use of bivalent anti-NKG2D antibodies. Since OMCP incorporated into our proposed bispecific construct is a monomer it cannot crosslink NKG2D. For this reason NKG2D activation by OMCP-containing bispecifics occurs only at the time of tumor engagement, i.e. once two or more tumor targeting domains engage the tumor ligand and bring the OMCP portion of the bispecific and their engaged NKG2D receptors into close proximity. Taken together both of these factors should provide a safety measure due to: 1) lack of non-specific and broad activation of naïve T cells that are not tumor reactive; 2) lack of T cell activation outside of the tumor bed; 3) lack of NKG2D crosslinking in the absence of tumor based ligand.

To evaluate lymphocyte activation and cytokine release cytokine production by bulk PBMCs incubated with EGFR-targeted bispecifics with different immune cell targeting domains: OMCP (E0), KYK1 anti-NKG2D scFv (E1), KYK2 anti-NKG2D scFv (E2), or OKT3 anti-CD3 scFv (E3) in the presence or absence of A549 tumor targets in vitro was studied.

500,000 freshly isolated peripheral blood mononuclear cells (PBMCs) (isolated from fresh blood by Ficoll enrichment) were plated in round bottom 96 well plates in 150 μl of media consisting of RPMI, 10% FCS and 1% penn/strep antibiotics. For some cultures 100,000 A549 lung cancer cells were added to the PBMCs while other PBMC cultures were left without tumor cells. EGFR-targeted bispecifics with different immune cell targeting domains: OMCP (E0), KYK1 anti-NKG2D scFv (E1), KYK2 anti-NKG2D scFv (E2), or OKT3 anti-CD3 scFv (E3) were then added to the cultures (both tumor containing and tumor non-containing) for a final concentration of either 10⁻⁶ M or 10⁻⁸ M. After 24 hours of culture the plate was spun down to concentrate the pellet and the cell free media was collected. Multiplex cytokine concentrations were measured using Luminex assay according to manufacturer protocols (ThermoFischer Scientific).

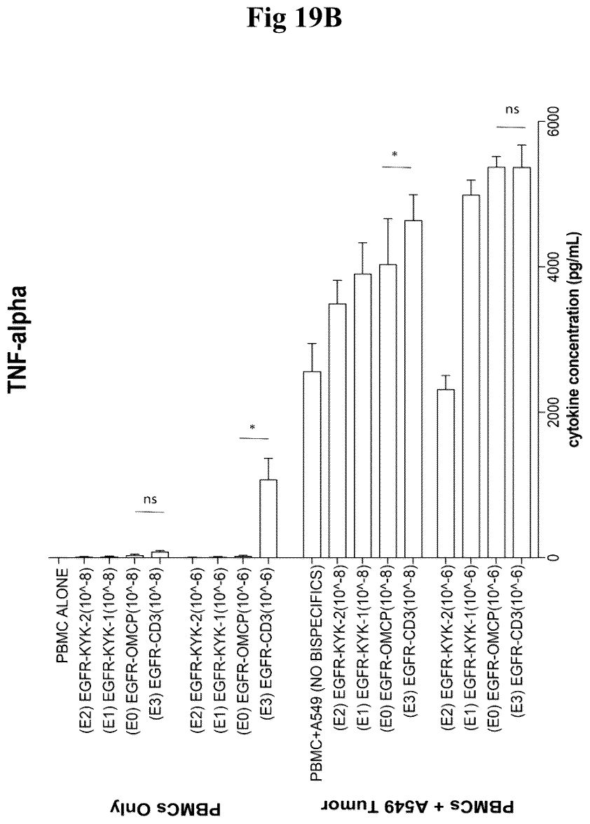

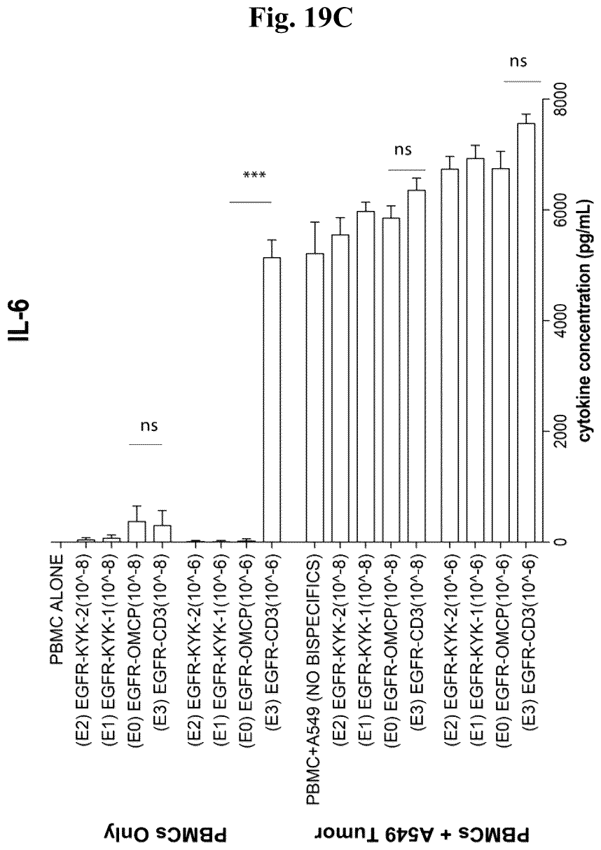

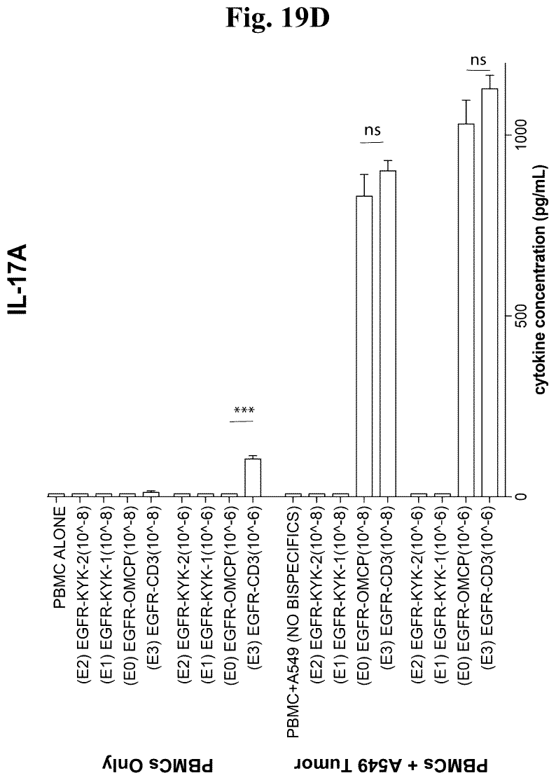

FIGS. 19A-19D show cytokine production for the various constructs tested at various concentrations with PMBCs alone or with PBMCs and tumor cells, using PMBCs alone and PBMCs with tumor cells as controls.

As demonstrated by FIGS. 19A-19D, E3 or the OKT3 containing construct results in serum cytokine release in the presence or absence of tumor cells supporting the non-specific global activation of cytotoxic lymphocytes as being responsible for the side effect of cytokine storm. E0 or OMCP containing constructs, on the other hand, result in serum cytokine release only when cultured with tumors. Such data supports our hypothesis that cytokine release and lymphocyte activation by E0 (OMCP containing bispecific constructs) occurs only at the site of the tumor and thus supports the notion that we should not obtain non-specific activation and thus could avoid the main side effect of bispecifics-cytokine release syndrome or cytokine storm (***=p<0.001; **p<0.01).

### Example 6—In-Vitro Cytotoxicity Assay

This example describes in vitro testing of OMCP-tumor targeted bispecific therapies. Specifically, this example will demonstrate improved human cytotoxic immune cell response against human target cell lines relevant to the bispecific tumor target.

Fresh human lymphocytes will be collected from donors, purified, and seeded with target cells in triplicate at the following ratios: no target cells, 15.6:1, 31.25:1, 62.5:1, 125:1, 250:1, 500:1. After 4 hours, the live versus dead target cell ratio will be evaluated via flow cytometry. Lymphocytes and target cells will be additionally incubated with the relevant protein construct at the following concentration: 10 μg/mL, 5 μg/mL, 1 μg/mL, 0.5 μg/mL, 0.1 μg/mL, or saline control. In addition to the constructs outlined in the table below, a fusion protein constructed from OMCP and a non-targeted antibody (OMCP-NT) will be tested against all cell lines.

Potential outcomes include finding that cytotoxic activity of freshly collected human PBMCs with tumors is enhanced by the presence of one or more OMCP-bispecific construct. Specifically, we expect to find that lymphocyte cytotoxic activity is increased proportionally to the expression of the antibody target on the tumor cell surface. Additionally, we expect to find that OMCP-NT neither enhances nor inhibits the functionality of the human lymphocytes against the target cells as compared to saline control.

The bispecific constructs and cell lines used will be as follows:

| Construct | Cell Line | Tissue of Origin |
|---|---|---|
| OMCP-anti-EGFR | HCC827 | Lung |
| | MDA-MB-468 | Breast |
| OMCP-anti-ERBB2 | HCC1954 | Breast |
| OMCP-anti-PMEL | HT-144 | Skin |
| | DU 145 | Prostate |
| OMCP-anti-CEACAM5 | PANC 05.04 | Pancreas |
| OMCP-anti-ERBB2- | HCC1954 | Breast |
| anti-EGFR | MDA-MB-468 | Breast |
| | HCC827 | Lung |

### Example 7—Melanoma Tumor Growth and Survival in Mice Treated With OMCP-Anti-PMEL or OMCP-Anti-EGFR

A total of 30 C57B1/6 mice 6-9 weeks of age will be utilized. Mice will be injected with B16 melanoma subcutaneously at the flank with 1×10⁶ cells per mouse. Treatment will begin 5 days later, when tumors have grown sufficiently to become visible and measurable. Initial tumor sizes and mouse weights will be taken, and mice will be randomized into groups of 10 mice such that the initial tumor sizes and mouse weights are similar between groups. The treatment groups will be as follows: Group 1—saline control, Group 2—OMCP-NT treatment, Group 3—OMCP-anti-PMEL treatment, Group 4—OMCP-anti-EGFR. All mice will be treated 2x weekly for 3weeks, a total of 5 doses. Group 1—The mice will be intraperitoneally (i.p.) administered 200 μL saline for all treatments as a negative control. Group 2—The mice will be i.p. administered 200 μg OMCP-NT in 200 μL saline. Group 3—The mice will be i.p. administered 200 μg OMCP-anti-PMEL in 200 μL saline. Group 4—The mice will be i.p. administered 200 μg OMCP-anti-EGFR in 200 μL saline.

All tumors will be measured via caliper measurements and mouse weights measured every day during treatment. After the completion of the therapeutic course, mouse weights and tumors will be measured thrice weekly. Mice will be monitored throughout the study for signs of distress or other effects of the therapeutic treatment. All mice will be euthanized at a maximum tumor diameter of 20 mm, and tumors will be reserved for later analysis. Any mice that die prematurely from known or unknown causes will have a final measurement taken and tissues collected as soon as is possible.

The B16 melanoma cell line was selected here due to the high level of expression of gp100 (PMEL) and EGFR. Therefore, potential outcomes may include a finding that treatment with OMCP-anti-PMEL will significantly attenuate tumor growth and increase survival times over the saline control group. We will further analyze residual tumors for lymphocyte infiltration via immunohistochemistry. Specifically, we will evaluate the intratumoral infiltration of CD8+ Teff cells and NK cells. Further, we will evaluate the apoptotic levels via a TUNEL assay (Millipore ApopTag Peroxidase In Situ Apoptosis Detection Kit, Cat No. S7100). Potential outcomes include a finding that treatment with PDL1-mutIL2 and PDL2-mutIL2 increases CD8+ Teff an NK cell intratumoral infiltration significantly over either saline control mice or wt IL2 treated mice.

These results would suggest that OMCP-anti-PMEL and OMCP-anti-EGFR have an enhanced therapeutic benefit against tumors with high target expression as compared to untreated mice. By using OMCP to engage both NK and CD8⁺ T cells specifically to the tumor surface, we expect to find that tumor infiltration and cytotoxic engagement for these key cell populations is significant.

### Example 8—Testing of Bi-Specific Fc Constructs

The following Fc constructs will be tested according to all of the foregoing examples as performed for the scFv constructs (E0, E1, E2, and E3).

| | SEQ ID | |
|---|---|---|
| Construct | NOs | Description |
| Asymmetric, bispecific | 31 & 32 | OMCP-Fc (KiH:Knob2) (SEQ ID NO: 31) |
| (OMCP, anti-EGFR) | | EGFR-Fc (KiH:Hole2) (SEQ ID NO: 32) |
| Symmetric, bispecific | 33 | OMCP-WT Fc-EGFR |
| (OMCP, anti-EGFR) | | |
| Single-arm asymmetric, | 34 & 35 | OMCP-EGFR-Fc (KiH:Knob2) (SEQ ID NO: |
| bispecific | | 34) |
| | | Fc (KiH:Hole2) (SEQ ID NO: 35) |
| Monomeric Fc, bispecific | 36 | OMCP-mFc-EGFR |

It should be understood that the foregoing description provides embodiments of the present invention which can be varied and combined without departing from the spirit of this disclosure. To the extent that the different aspects disclosed can be combined, such combinations are disclosed herein.

1-69. (canceled)

70. A polypeptide comprising a first domain and a second domain, wherein the first domain comprises a first amino acid sequence that possesses at least 80% homology to amino acid positions 48 to 67 and 110 to 147 of SEQ ID NO: 1, at least 80% homology to amino acid positions 49 to 68 and 111 to 148 of SEQ ID NO: 2, or at least 80% homology to amino acid positions 48 to 66 and 111 to 148 of SEQ ID NO: 3, wherein the first domain is capable of binding to human NKG2D with a binding affinity of about 0.01 nM to about 1000 nM, wherein the second domain comprises a second amino acid sequence capable of binding to a peptide on a tumor cell, wherein the peptide is either specific to the tumor cell or overexpressed on the tumor cell compared to a non-tumor cell of the same tissue origin as the tumor cell, and wherein the peptide is selected from the group consisting of ERBB2, CD19, EPCAM, MS4A1, FOLH1, CEACAM5, PMEL, CLEC12A, KDR, EGFR, TAG-72 (tumor associated glycoprotein 72), disialoganglioside GD2, CD20, CD123, CD33, BCMA, CD38, B7H3/CD276, GPA33, SSTR2, GPC3, and CDH30.

71. The polypeptide of claim 70, wherein the second domain is an antibody.

72. The polypeptide of claim 70, wherein the tumor cell is selected from the group consisting of a breast cancer cell, a prostate cancer cell, a melanoma cell, an ovarian cancer cell, a gastric cancer cell, a glioblastoma cell, a neuroblastoma cell, a lung cancer cell a lymphoma cell, a leukemia cell, a colon cancer cell, a renal cell carcinoma, a pancreatic cancer cell, and a hepatocellular carcinoma cell.

73. The polypeptide of claim 70, wherein the first amino acid sequence possesses at least 90% homology to amino acid positions 48 to 67 and 110 to 147 of SEQ ID NO: 1, at least 90% homology to amino acid positions 49 to 68 and 111 to 148 of SEQ ID NO: 2, and at least 90% homology to amino acid positions 48 to 66 and 111 to 148 of SEQ ID NO: 3.

74. The polypeptide of claim 70, wherein the first amino acid sequence possesses at least 80% homology to SEQ ID NOs: 1, 2 or 3.

75. A pharmaceutical composition comprising a polypeptide and a pharmaceutically acceptable excipient, wherein the polypeptide comprises a first domain and a second domain, wherein the first domain comprises a first amino acid sequence that possesses at least 80% homology to amino acid positions 48 to 67 and 110 to 147 of SEQ ID NO: 1, at least 80% homology to amino acid positions 49 to 68 and 111 to 148 of SEQ ID NO: 2, or at least 80% homology to amino acid positions 48 to 66 and 111 to 148 of SEQ ID NO: 3, wherein the first domain is capable of binding to human NKG2D with a binding affinity of about 0.01 nM to about 1000 nM, wherein the second domain comprises a second amino acid sequence capable of binding to a peptide on a tumor cell, wherein the peptide is either specific to the tumor cell or overexpressed on the tumor cell compared to a non-tumor cell of the same tissue origin as the tumor cell, and wherein the peptide is selected from the group consisting of ERBB2, CD19, EPCAM, MS4A1, FOLH1, CEACAM5, PMEL, CLEC12A, KDR, EGFR, TAG-72 (tumor associated glycoprotein 72), disialoganglioside GD2, CD20, CD123, CD33, BCMA, CD38, B7H3/CD276, GPA33, SSTR2, GPC3, and CDH30.

76. The pharmaceutical composition of claim 75, wherein the second domain is an antibody.

77. The pharmaceutical composition of claim 75, wherein the tumor cell is selected from the group consisting of a breast cancer cell, a prostate cancer cell, a melanoma cell, an ovarian cancer cell, a gastric cancer cell, a glioblastoma cell, a neuroblastoma cell, a lung cancer cell, a lymphoma cell, a leukemia cell, a colon cancer cell, a renal cell carcinoma, a pancreatic cancer cell, and a hepatocellular carcinoma cell.

78. The pharmaceutical composition of claim 75, wherein the first amino acid sequence possesses at least 90% homology to amino acid positions 48 to 67 and 110 to 147 of SEQ ID NO: 1, at least 90% homology to amino acid positions 49 to 68 and 111 to 148 of SEQ ID NO: 2, and at least 90% homology to amino acid positions 48 to 66 and 111 to 148 of SEQ ID NO: 3.

79. The pharmaceutical composition of claim 75, wherein the first amino acid sequence possesses at least 80% homology to SEQ ID NOs: 1, 2 or 3.

80. The pharmaceutical composition of claim 75, wherein the pharmaceutically acceptable excipient is selected from the group consisting of a diluent, a binder, a filler, a buffering agent, a pH modifying agent, a disintegrant, a dispersant, a preservative, a lubricant, a taste-masking agent, a flavoring agent, and a coloring agent.

81. A polypeptide comprising a first domain and a second domain, wherein the first domain comprises a first amino acid sequence that possesses at least 80% homology to amino acid positions 48 to 67 and 110 to 147 of SEQ ID NO: 1, at least 80% homology to amino acid positions 49 to 68 and 111 to 148 of SEQ ID NO: 2, or at least 80% homology to amino acid positions 48 to 66 and 111 to 148 of SEQ ID NO: 3, wherein the first domain is capable of binding to human NKG2D with a binding affinity of about 0.01 nM to about 1000 nM, wherein the second domain comprises a second amino acid sequence capable of binding to a peptide on a tumor cell, wherein the peptide is either specific to the tumor cell or overexpressed on the tumor cell compared to a non-tumor cell of the same tissue origin as the tumor cell, wherein the peptide is selected from the group consisting of ERBB2, CD19, EPCAM, MS4A1, FOLH1, CEACAM5, PMEL, CLEC12A, KDR, EGFR, TAG-72 (tumor associated glycoprotein 72), disialoganglioside GD2, CD20, CD123, CD33, BCMA, CD38, B7H3/CD276, GPA33, SSTR2, GPC3, and CDH30, and wherein the second domain is not a cytokine.

82. A method for treating a tumor in a subject in need thereof comprising administering to the subject in need thereof a therapeutically effective amount of a pharmaceutical composition, the pharmaceutical composition comprising a polypeptide and a pharmaceutically acceptable excipient, wherein the polypeptide comprises a first domain and a second domain, wherein the first domain comprises a first amino acid sequence that possesses at least 80% homology to amino acid positions 48 to 67 and 110 to 147 of SEQ ID NO: 1, at least 80% homology to amino acid positions 49 to 68 and 111 to 148 of SEQ ID NO: 2, or at least 80% homology to amino acid positions 48 to 66 and 111 to 148 of SEQ ID NO: 3, wherein the first domain is capable of binding to human NKG2D with a binding affinity of about 0.01 nM to about 1000 nM, wherein the second domain comprises a second amino acid sequence capable of binding to a peptide on a tumor cell, wherein the peptide is either specific to the tumor cell or overexpressed on the tumor cell compared to a non-tumor cell of the same tissue origin as the tumor cell, and wherein the peptide is selected from the group consisting of ERBB2, CD19, EPCAM, MS4A1, FOLH1, CEACAM5, PMEL, CLEC12A, KDR, EGFR, TAG-72 (tumor associated glycoprotein 72), disialoganglioside GD2, CD20, CD123, CD33, BCMA, CD38, B7H3/CD276, GPA33, SSTR2, GPC3, and CDH30.

83. The method of claim 82, wherein the second domain is an antibody.

84. The method of claim 82, wherein the tumor cell is selected from the group consisting of a breast cancer cell, a prostate cancer cell, a melanoma cell, an ovarian cancer cell, a gastric cancer cell, a glioblastoma cell, a neuroblastoma cell, a lung cancer cell, a lymphoma cell, a leukemia cell, a colon cancer cell, a renal cell carcinoma, a pancreatic cancer cell, and a hepatocellular carcinoma cell.

85. The method of claim 82, wherein the first amino acid sequence possesses at least 90% homology to amino acid positions 48 to 67 and 110 to 147 of SEQ ID NO: 1, at least 90% homology to amino acid positions 49 to 68 and 111 to 148 of SEQ ID NO: 2, and at least 90% homology to amino acid positions 48 to 66 and 111 to 148 of SEQ ID NO: 3.

86. The method of claim 82, wherein the first amino acid sequence possesses at least homology to SEQ ID NOs: 1, 2 or 3.

87. The method of claim 82, wherein the pharmaceutically acceptable excipient is selected from the group consisting of a diluent, a binder, a filler, a buffering agent, a pH modifying agent, a disintegrant, a dispersant, a preservative, a lubricant, a taste-masking agent, a flavoring agent, and a coloring agent.

88. The method of claim 82, wherein the step of administering is performed orally or otherwise peripherally.

89. The method of claim 82, wherein the step of administering is peripherally and is selected from the group consisting of intravenous, intraperitoneal, subcutaneous, pulmonary, transdermal, intramuscular, intranasal, buccal, sublingual, or suppository.

90. The method of claim 82, wherein the step of administering is performed by subcutaneous, intramuscular, or intravenous injection.
